SEQUENCE LISTING (9 sequences)

SEQ ID NO: 1 - protein, 14 aa, Homo sapiens
TQMGSPSVRCSAMA

SEQ ID NO: 2 - protein, 30 aa, Homo sapiens
RQIKIWFQNRRMKWKKTQMGSPSVRCSAMA

SEQ ID NO: 3 - protein, 30 aa, Homo sapiens
RQIKIWFQNRRMKWKKTQMGSPSVRCSSMS

SEQ ID NO: 4 - protein, 30 aa, Homo sapiens
RQIKIWFQNRRMKWKKTQMGSPLNPISAVA

SEQ ID NO: 5 - protein, 16 aa, Homo sapiens
RQIKIWFQNRRMKWKK

SEQ ID NO: 6 - protein, 14 aa, Homo sapiens
TQMGSPSVRCSSMS

SEQ ID NO: 7 - protein, 14 aa, Homo sapiens
  MISC_FEATURE (12)..(12): Xaa is not Ser and preferably is not Thr
TQMGSPSVRCSXMX

SEQ ID NO: 8 - protein, 30 aa, Homo sapiens
  MISC_FEATURE (28)..(28): Xaa is not Ser and preferably is not Thr
RQIKIWFQNRRMKWKKTQMGSPSVRCSXMX

SEQ ID NO: 9 - protein, 14 aa, Homo sapiens
TQMGSPSIRCSSVS

(19) United States
(12) Patent Application Publication
(10) Pub. No.: US 2005/0136043 A1
(43) Pub. Date: Jun. 23, 2005

(54) TGFbeta signaling inhibitors

(75) Inventors: Ihor Yakymovych, Uppsala (SE); Ulla Engstrom, Uppsala (SE); Susanne Grimsby, Uppsala (SE); Carl-Henrik Heldin, Uppsala (SE); Serhiy Souchelnytskyi, Uppsala (SE)

Correspondence Address: HAUPTMAN KANESAKA BERNER PATENT AGENTS, SUITE 300, 1700 DIAGONAL RD, ALEXANDRIA, VA, 22314-2848, US

(73) Assignee: Ludwig Institute for Cancer Research, New York, NY (US)

(21) Appl. No.: 10/911,414

(22) Filed: Aug. 4, 2004

Related U.S. Application Data
(60) Provisional application No. 60/493,135, filed on Aug. 7, 2003.

(51) Int. Cl. A61K 38/43; C12N 9/99

(52) U.S. Cl. 424/94.1; 435/184

(57) ABSTRACT

This invention relates to peptide molecules and compounds which are negative regulators of TGFβ signaling, particularly of TβR-I kinase activity. The invention further relates to methods of using such peptides and compounds in the treatment of disease.

### RELATED APPLICATION

This application claims the benefit under 35 U.S.C. 119(e) of U.S. provisional patent application Ser. No. 60/493,135, filed Aug. 7, 2003, the contents of which are incorporated herein by reference in their entirety.

### FIELD OF THE INVENTION

This invention relates to peptide molecules and compounds which are negative regulators of TGFβ signaling. The invention further relates to methods of using such peptides and compounds in the treatment of disease.

### BACKGROUND OF THE INVENTION

During mammalian embryonogenesis and adult tissue homeostasis, transforming growth factor-β (TGFβ) performs pivotal tasks in intercellular communications [1]. TGFβ is a polypeptide growth factor involved in regulation of cell proliferation, differentiation, apoptosis and migration [1, 2]. Two types of TGFβ-specific serine/threonine kinase receptors, type I and type II (TβR-I and TβR-II), are essential for the TGFβ signaling [3]. Upon interaction with the ligand, a homodimer of TβR-II recruits two TβR-I molecules and activates their kinases by phosphorylation of serine residues in the GS region of TβR-I [4, 5]. In this heterotetrameric complex, activated TβR-I phosphorylates Smad proteins, which are major components of the TGFβ intracellular signaling pathway [6, 7].

TGFβ is involved in pathogenesis of many diseases, including cancer, as well as fibrotic and immunological disorders. TGFβ signaling has a tumor promoting effect at late stages of tumorigenesis, when malignant cells have lost responsiveness to the growth inhibitory action of TGFβ, via action on non-malignant cells surrounding the tumor cells, such as immune cells, endothelial cells and connective tissue cells [8, 9]. An excessive activation of TGFβ signaling can also cause fibrotic disorders, as TGFβ is a potent stimulator of extracellular matrix formation [10]. TGFβ1 null mice die due to a severe wasting syndrome, which is in agreement with the potent immunosuppressor action of TGFβ [11]. Tools to regulate TGFβ signaling would be warranted to selectively modulate TGFβ-dependent processes in various medical conditions.

TβR-I kinase can be regulated by interaction with other proteins or by phosphorylation [12]. Phosphorylation of TβR-1 in the juxtamembrane GS-region by TβR-II is crucial for its activation, whereas TβR-I-interacting proteins have a modulatory effect on TGFβ signaling [3-5, 12]. Thus, an efficient way to affect TGFβ signaling, is by affecting the kinase activity of TβR-I.

Low molecular weight compounds have been used as potent inhibitors of tyrosine kinases as well as serine/threonine kinases [13]. Most of these inhibitors block the ATP-binding sites of the respective enzymes. Despite significant similarity of the ATP-binding sites in kinases, it has been possible to develop inhibitors a high degree of selectivity. However, absolute specificities have not been achieved which complicates their use in treatment of diseases [13, 14]. Inhibitors acting through binding to the ATP-binding site often suffer from loss of specificity, because ATP-binding sites in all studied kinases share significant similarity. Search for specific inhibitors of kinases involved in intracellular signaling, is an important task in the development of drugs, because it may target selected regulatory pathways.

### SUMMARY OF THE INVENTION

The invention is based, in part, on the discovery that Sm2 peptide molecules corresponding to the C-terminal amino acids of Smad2 act as substrate mimicking peptides or pseudosubstrates that inhibit TGFβ signaling and Smad2 phosphorylation. In view of this discovery, the invention provides isolated peptides and methods of using such peptides for the treatment of disease.

According to one aspect of the invention, isolated peptides that inhibit TGFβ signaling and/or Smad2 phosphorylation are provided. The isolated peptides include the amino acid sequence of SEQ ID NO:6 or SEQ ID NO:7. In certain embodiments, the isolated peptide comprises SEQ ID NO:7. Preferably, the isolated peptide consists of SEQ ID NO:7. In other embodiments, the isolated peptide comprises SEQ ID NO: 1. Preferably, the isolated peptide consists of SEQ ID NO: 1. In still other embodiments, the isolated peptide comprises SEQ ID NO:6. Preferably, the isolated peptide consists of SEQ ID NO:6.

In certain embodiments, the foregoing peptides are conjugated to a molecule that facilitates in vitro or in vivo penetration of the peptide into cells. Preferably the molecule that facilitates cell penetration is a peptide. In a particularly preferred embodiment, the peptide that facilitates cell penetration is a penetratin peptide of antennapedia (SEQ ID NO:5).

Preferably the the peptide that facilitates cell penetration is a fusion peptide with the isolated peptide. In such embodiments the peptide conjugate comprises the amino acid sequence set forth as SEQ ID NO:2, SEQ ID NO:3, or SEQ ID NO:8.

According to another aspect of the invention, isolated peptide functional variants are provided. The functional variants include an amino acid sequence having 1-5 amino acid additions, substitutions, or deletions of of SEQ ID NO:1, SEQ ID NO:6 or SEQ ID NO:7, wherein the peptide functional variant inhibits TGFβ signaling (particularly via inhibiting TβR-I kinase activity) and/or Smad2 phosphorylation.

In certain embodiments, the foregoing peptides are conjugated to a molecule that facilitates in vitro or in vivo penetration of the peptide into cells. Preferably the molecule that facilitates cell penetration is a peptide. In a particularly preferred embodiment, the peptide that facilitates cell penetration is a penetratin peptide of antennapedia (SEQ ID NO:5).

Preferably the the peptide that facilitates cell penetration is a fusion peptide with the isolated peptide. In preferred embodiments the peptide conjugate includes the amino acid sequence set forth as SEQ ID NO:2, SEQ ID NO:3, or SEQ ID NO:8.

In still another aspect of the invention, compositions including the foregoing peptides or functional variants and a pharmaceutically acceptable carrie are provided. In certain embodiments, such compositions include one or more molecules selected from the group consisting of: SB203580, SB202190, SB202474, PD169316, and SC68376.

Also provided are methods for making a medicament, which include placing a therapeutically effective amount of the isolated peptids or functional variants in a pharmaceutically acceptable carrier. In certain embodiments, the methods also include adding a therapeutically effective amount of one or more molecules selected from the group consisting of: SB203580, SB202190, SB202474, PD169316, and SC68376.

According to another aspect of the invention, methods of inhibiting TGFβ signaling in a cell or cell extract are provided. The methods include contacting a cell or cell extract having TGFβ signaling with an effective amount of the foregoing peptides or functional variants to inhibit TGFβ signaling in the cell or cell extract. Additional method include contacting a cell or cell extract having TGFβ signaling with an effective amount of (a) the foregoing peptides or functional variants and (b) one or more molecules selected from the group consisting of: SB203580, SB202190, SB202474, PD169316, and SC68376, to inhibit TGFβ signaling in the cell or cell extract.

Similar methods are provided for inhibiting Smad2 phosphorylation in a cell or cell extract

In certain embodiments of the foregoing methods, the TGFβ signaling is inhibited in vivo in a cell. In other embodiments of the foregoing methods, the TGFβ signaling is inhibited in vitro. In preferred embodiments, TβR-I kinase activity is inhibited.

According to still another aspect of the invention, methods of treating a subject having or at risk of having an increased TGFβ signaling disorder are provided. The methods include administering to a subject in need of such treatment an effective amount of the foregoing peptides or functional variants to treat or prevent the increased TGFβ signaling disorder. Other methods of treating a subject having or at risk of having an increased TGFβ signaling disorder include administering to a subject in need of such treatment an effective amount of (a) the foregoing peptides or functional variants and (b) one or more molecules selected from the group consisting of: SB203580, SB202190, SB202474, PD169316, and SC68376, to treat the increased TGFβ signaling disorder. In these methods the increased TGFβ signaling disorder is selected from the group consisting of: cancer, fibrotic disorders, wound healing and immune disorders resulting from the immunosuppressor action of TGFβ.

Methods of treating a subject having or at risk of having a disorder that manifests increased TβR-I kinase activity are provided in another aspect of the invention. The methods include administering to a subject in need of such an effective amount of the foregoing peptides or functional variants to treat or prevent the increased TβR-I kinase activity disorder. Other methods of treating a subject having or at risk of having a disorder that manifests increased TβR-I kinase activity include administering to a subject in need of such treatment an effective amount of (a) the foregoing peptides or functional variants and (b) one or more molecules selected from the group consisting of: SB203580, SB202190, SB202474, PD169316, and SC68376, in an effective amount to treat the increased TβR-I kinase activity disorder.

In yet another aspect of the invention, methods of treating a subject having or at risk of having a disorder that manifests increased Smad2 phosphorylation are provided. The methods include administering to a subject in need of such an effective amount of the foregoing peptides or functional variants to treat or prevent the increased Smad2 phosphorylation disorder. Other methods of treating a subject having or at risk of having a disorder that manifests increased Smad2 phosphorylation include administering to a subject in need of such treatment an effective amount of (a) the foregoing peptides or functional variants and (b) one or more molecules selected from the group consisting of: SB203580, SB202190, SB202474, PD169316, and SC68376, in an effective amount to treat the increased Smad2 phosphorylation disorder.

Use of the foregoing peptides or functional variants in the preparation of medicaments, particularly for the disorders indicated herein, also is provided.

These and other aspects of the invention will be described in further detail in connection with the detailed description of the invention. Each of the limitations of the invention can encompass various embodiments of the invention. It is therefore, anticipated that each of the limitation involving any one element or combination of elements can be included in each aspect of the invention.

### BRIEF DESCRIPTION OF THE DRAWINGS

FIG. 1 shows that GST-TβR-I kinase preserves specificity for substrate phosphorylation. A representative experiment out of three performed is shown. FIG. 1A is a digital phosphorimager image of a SDS-PAGE gel showing the migration positions of GST-TβR-I, GST-Smad3deltaMH1, GST-Smad2 and GST-Smad1 after an in vitro kinase reaction. A representative experiment out of three performed is shown. FIG. 1B shows the results of tryptic digestion and two-dimensional phosphopeptide mapping of GST-TβR-I. Autophosphorylated GST-TβR-I, shown in lane 1 of panel A, and wild-type TβR-I activated by addition of TGFβ1 were subjected to tryptic digestion and two-dimensional phosphopeptide mapping. Arrowheads indicate the position of phosphopeptides observed in maps of GST-TβR-I (upper panel) and wild-type TβR-I (lower panel) (5). FIG. 1C shows two-dimensional phosphopeptide mapping of GST-Smad3 phosphorylated by GST-TβR-I. GST-Smad3deltaMH1, shown in lane 2 of panel A, and wild-type Smad3 activated by treatment of Mv1Lu cells with TGFβ1 were subjected to two-dimensional phosphopeptide mapping. Arrows show migration positions of the C-terminal peptide with single or double (left spot) phosphorylation on maps of GSTSmad3 (upper panel) and wild-type Smad3 (lower panel) (25). The arrowhead shows the migration of the linker-derived peptide with multiple phosphorylations. Sample application points in panels B and C are shown by triangles. Pi indicates the migration position of inorganic phosphate.

FIG. 2 depicts inhibition of GST-TβR-I kinase by inhibitors interfering with ATP-binding. Kinase activity of GST-TβR-I was evaluated by phosphorylation of the GST-Smad3deltaMH 1 in the in vitro kinase assay. The percent inhibition was calculated as 100×(1-Aᵢ/A₀), where Aᵢ and A₀ are the levels of phosphorylation in the presence or absence of the inhibitor, respectively. Kinase inhibitors representing different structural groups (FIG. 2A) and inhibitors of the pyridinylimidazole group (FIG. 2B) were prepared according to manufacturer's recommendations. Inhibitors were added in final concentrations as indicated. Samples were subjected to SDS-PAGE, and radioactivity incorporated in GST-Smad3deltaMH1 was evaluated by using Fuji X2000 phosphorimager. A representative experiment out or three performed is shown.

FIG. 3 shows that SB203580 is a potent inhibitor of TβR-I signaling. Representative experiments out of three performed are shown. FIG. 3A depicts phosphorylation of Smad2 in Mv1Lu cells stimulated with TGFβ1 in the presence or absence of inhibitors. FIG. 3B shows that SB203580 inhibited TGFβ-dependent activation of the luciferase reporter CAGA(12)-luc in Mv1Lu cells.

FIG. 4 depicts inhibition of GST-TβR-I kinase by substrate-mimicking peptides. Phosphorylated GST-TβR-I and GST-Smad2 were visualized after SDS-PAGE and exposure in FujiX2000 phosphorimager. FIG. 4A shows that Sm2 peptides inhibited autophosphorylation of GST-TβR-I in an in vitro kinase assay. GST-TβR-I autophosphorylation assay was performed in the absence or presence of antp-Sm2S, antp-Sm2A, and antp-Sm5A peptides at concentrations of 1 and 10 μM, as indicated. Arrows show the migration of GST-TβR-I. FIG. 4B depicts the inhibitory effect of Sm2 peptides on the phosphorylation at the C-terminus of GST-Smad2. Phosphorylation of GST-Smad2 by GST-TβR-I was performed in an in vitro kinase assay. The peptides were added as indicated. Arrows show the migration position of GST-Smad2 (GST-Sm2). Phosphorylated GST-TβR-I and GST-Smad2 were visualized after SDS-PAGE and exposure in a Fuji X2000 phosphorimager. The gels stained with Coomassie blue are shown to control equal loading. Representative experiments out of three (A) or two (B) performed are shown.

FIG. 5 shows that substrate-mimicking peptides inhibit TβR-I receptor kinase but have no effect on other type I and type II receptors of the TGFβ family and do not affect p38R and SAPKR2 kinase activities. The receptors and kinases were expressed in COS-7 cells and purified by precipitation with anti-tag antibodies or Ni-NTA agarose. Type I receptors [ALK-1, ActR-I (ALK-2), BMPR-IA (ALK-3), ActR-IB (ALK- 4), TβR-I (ALK-5), BMPR-IB (ALK-6), and ALK-7] were HA-tagged (FIG. 5A). TβR-II, ActR-II, and BMPR-II were His6-tagged (FIG. 5B), and p38R and SAPKR2 were HA-tagged (FIG. 5C). In vitro kinase assays were performed in the presence of 10 μM peptides or not, as indicated. Equal loading and expression of kinases were controlled by immunoblotting with anti-tag antibodies (lower panels). Migration positions of kinases on autoradiographs and immunoblots are indicated. A representative experiment out of two performed is shown.

FIG. 6 shows that substrate-mimicking peptides inhibited TGFβ signaling as determined by TGFβ1-dependent phosphorylation of endogenous Smad2 (FIG. 6A) or activation of a luciferase reporter under control of a Smad2-responsive element (FIG. 6B). Representative experiments out of three (FIG. 6A) or two (FIG. 6B) performed, are shown. *p<0.05, cells pre-treated with antp-Sm2A (SEQ ID NO: 2) peptide before addition of TGFβ1 compared to cells treated with TGFβ1 only.

FIG. 7 shows that substrate-mimicking peptides revert TGFβ-dependent inhibition of DNA synthesis. Mv1Lu cells were pretreated with peptides at a final concentration of 50 μM and incubated with TGFβ1 (0.1 ng/mL) or not for 24 h, as indicated. [³H]Thymidine was added to cells for the last 2 h of incubation, and radioactivity incorporated into DNA was measured. A representative experiment out of four performed is shown. *, p <0.05; cells pretreated with antp-Sm2A or antp-Sm2S peptides were compared to cells pretreated with antp peptide.

FIG. 8 is a schematic presentation of residues which may interact with SB203580 in p38 and TβR-I kinases. Residues which differ are shown in bold.

### DETAILED DESCRIPTION OF THE INVENTION

Transforming growth factor-β (TGFβ) is a potent regulator of cell proliferation, differentiation, apoptosis and migration. TGF-β type I receptor (TβR-I), which has intrinsic serine/threonine kinase activity, is a key component in activation of intracellular TGFβ signaling. TβR-I kinase can be regulated by interaction with other proteins or by phosphorylation [12].

Low molecular weight compounds have been used as potent inhibitors of tyrosine kinases as well as serine/threonine kinases [13]. Although it has been possible to develop small molecule inhibitors with a high degree of selectivity of the ATP-binding sites in kinases, absolute specificities have not been achieved, which complicates the use of such small molecule inhibitors in treatment of diseases [13, 14].

The invention is based in part on the discovery that peptides mimicking the TβR-I phosphorylation sites at the C-terminus of Smad2 also inhibited the autophosphorylation of TβR-I and phosphorylation of Smad2 by TβR-I in vitro and in vivo. The inhibition of TβR-I occurs with unexpected specificity. Surprisingly, as shown below, the activity of other kinases (ActR-IB (ALK-4) and ALK-7) that also phosphorylate Smad2 can be affected by the C-terminus peptides of Smad2, as was TβR-I, but with a different affinity for the peptide.

TβR-I kinase activates signaling by phosphorylation of Smad2 and Smad3. The phosphorylation of these two Smads has similar mechanism, with phosphorylation of C-terminal serine residues. The inhibition of TβR-I (e.g., blocking kinase activity) as shown herein permits the inhibition of phosphorylation of both Smad2 and Smad3, and inhibition of TGFβ signaling. In addition, phosphorylation of other substrates than Smads by TβR-I kinase also can be inhibited using the methods and compositions of the invention. Thus, substrate-mimetic peptides are a new type of specific inhibitors of the TGFβ signaling in vivo, which permit specific inhibition of TβR-I kinase activity. In view of the foregoing discoveries, the following inventions are provided.

Sm2 peptides, as used herein, are substrate mimicking inhibitors of T βR-I. An isolated Sm2 peptide, as used herein, includes the amino acid sequence of the C-terminus of Smad2, preferably the fragment represented by SEQ ID NO:6, or a functional variant thereof. Preferably, the isolated Sm2 peptide consists of SEQ ID NO:6, or a functional variant thereof. In particularly preferred embodiments, the Sm2 peptide is a functional variant that has substitutions at phosphorylatable serines of the C-terminus of Smad2 (SEQ ID NO:7, wherein X is not Ser and preferably not Thr). Preferably, the substitutions (X residues) are alanine (e.g., SEQ ID NO: 1). In yet other embodiments, the isolated Sm2 peptide includes or consists of a substituted or an unsubstituted fragment of the C-terminus of Smad2 attached to a molecule that facilitates in vitro or in vivo penetration of the Sm2 peptide into cells. A preferred example of such a molecule, is a penetratin peptide of antennapedia (SEQ ID NO:5). Thus, preferred Sm2 peptides include substituted (e.g., SEQ ID NO: 2) or unsubstituted (e.g., SEQ ID NO: 3) Smad2 C-terminal fragments attached to penetratin peptide. A generic representation of a substituted Sm2 peptide attached to penetratin peptide is SEQ ID NO:8. Furthermore, Sm2 peptides include the similar region of the Smad3 C-terminus (SEQ ID NO:9), along with mutated variants having substitutions of Ser residues as in the Smad2 C-terminus peptides (e.g., Ser12 and Ser14 of SEQ ID NO:9), penetratin fusion peptides, and functional variant thereof.

A functional variant of a Sm2 peptide is a peptide that has 1-5 amino acid additions, substitutions, or deletions of SEQ ID NO:1, 6 or 7, but which retains the function of inhibiting TβR-I activity.

The term “peptide,” as used herein encompasses the terms polypeptide and protein. As used herein, a “Sm2 peptide” refers to a peptide having the activity of a native Sm2 peptide such as that described by SEQ ID NO:6. Thus, for example a Sm2 peptide includes peptides which include the amino acids of SEQ ID NO:6, and functional variants thereof (e.g., SEQ ID NO:7, preferably SEQ ID NO:1) provided that the functional variant exhibits a Sm2 peptide functional activity. As used herein, a “Sm2 peptide functional activity” includes the ability of a Sm2 peptide to inhibit TGFβ signaling; growth inhibition; inhibition of TβR-I kinase activity -dependent disorders, including Smad2 phosphorylation; tumor suppression; reduction of tumor cell growth, proliferation, and/or metastasis; inhibition of fibrosis; and inhibition of immunological disorders.

Sm2 peptide functional activity can be determined in various in vitro assays. For example, the modulation of TGFβ signaling can be determined by measuring the effect of a Sm2 peptide on TGFβ receptor autophosphorylation, as is shown in the Examples below. The inhibition of TβR-I kinase activity -dependent disorders, including Smad2 phosphorylation, by Sm2 peptides also can be determined in an in vitro assay using Smad2 (or a fragment thereof containing a TGFβ receptor phosphorylation site) in combination with TGF, receptor, with or without a candidate Sm2 peptide.

Sm2 peptide functional activity also can be determined by assaying tumor growth in an organism. According to this embodiment, the assay involves: measuring the tumor size in an organism before treatment with a Sm2 peptide, measuring the tumor size in the organism after treatment with a Sm2 peptide, and comparing the tumor size in the organism before and after treatment with the Sm2 peptide. Alternatively, the growth of tumor cells can be measured as an in vitro measure of Sm2 peptide functional activity. In these embodiments, a decrease or no increase in the tumor size (or growth of tumor cells) after treatment with the Sm2 peptide indicates that the Sm2 peptide has a Sm2 peptide functional activity. A decrease in the rate of tumor growth (or a decrease in the rate of tumor cell growth) also indicates Sm2 peptide functional activity. An increase in the tumor size or growth of tumor cells without a decrease in the rate of growth of the tumor or cells after treatment with the Sm2 peptide indicates that the Sm2 peptide does not have a Sm2 peptide functional activity.

Sm2 peptide functional activity also can be determined in a subject by measuring tumor size before and after treatment with a Sm2 peptide, and comparing the results. Similarly, Sm2 peptide functional activity can be determined by measuring the number of tumor cells in a sample obtained from a subject before and after treatment, or sequentially over time during the course of treatment. Functional activity is found when there is a decrease or no increase in the tumor size (or number of tumor cells), or when there is a reduction in the growth rate of the tumor relative to an untreated control.

The activity of small molecule TGFβ inhibitors having a pyridinylimidazole group is tested using similar methods, some of which are shown in the Examples below.

Sm2 peptides can be isolated from biological samples including tissue or cell homogenates, but preferably are expressed recombinantly using a prokaryotic or eukaryotic expression system by constructing an expression vector appropriate to the expression system, introducing the expression vector into the expression system, incubating the expression system for a time sufficient to express the Sm2 peptide, and isolating the recombinantly expressed peptide. Peptides also can be synthesized chemically using well-established methods of peptide synthesis.

Thus, as used herein with respect to peptides, “isolated” means separated from its native environment and present in sufficient quantity to permit its identification or use. Isolated, when referring to a peptide means, for example: (i) selectively produced by expression of a recombinant nucleic acid or (ii) purified as by chromatography or electrophoresis. Isolated peptides may, but need not be, substantially pure. The term “substantially pure” means that the peptides are essentially free of other substances with which they may be found in nature or in vivo systems to an extent practical and appropriate for their intended use. Substantially pure peptides may be produced by techniques well known in the art. Because an isolated peptide may be admixed with a pharmaceutically acceptable carrier in a pharmaceutical preparation, the peptide may comprise only a small percentage by weight of the preparation. The peptide is nonetheless isolated in that it has been separated from the substances with which it may be associated in living systems, e.g. isolated from other peptides.

The invention embraces variants of the Sm2 peptide described herein. As used herein, a “variant” of a Sm2 peptide is a peptide which contains one or more modifications to the primary amino acid sequence of a Sm2 peptide. Modifications include deletions, point mutations, truncations, amino acid substitutions and additions of amino acids or non-amino acid moieties. Modifications which create a Sm2 peptide variant can be made to a Sm2 peptide 1) to provide equivalent or better Sm2 peptide functional activity such as, for example, stronger inhibition of TGFβ signaling; 2) to enhance a property of the Sm2 peptide, such as peptide stability in an expression system or the stability of peptide binding; 3) to provide a novel activity or property to a Sm2 peptide.

When produced in an expression system,modifications to a Sm2 peptide can be made to the nucleic acid which encodes a Sm2 peptide. When a Sm2 peptide is synthesized, modifications can be made directly to the peptide. Further modifications made directly to a Sm2 peptide include cleavage, addition of a linker molecule, addition of a detectable moiety, such as biotin, addition of a fatty acid, and the like. Modifications also embrace fusion proteins comprising all or part of the Sm2 peptide amino acid sequences.

One of skill in the art will be familiar with methods for predicting the effect on peptide conformation of a change in amino acid sequence, and can thus “design” a variant Sm2 peptide according to known methods. One example of such a method is described by Dahiyat and Mayo in Science 278:82-87, 1997, whereby peptides can be designed de novo. Specific variants of a Sm2 peptide can be proposed and tested to determine whether the variant retains a desired conformation. Alternatively, libraries of variant peptides can be prepared by substitution, addition or deletion of one or more amino acids (preferably 1-5 amino acids) These libraries can be tested to determine a selected activity of the peptides in the libraries, and peptides of suitable activity can be selected for further testing or use. Such methods can be applied to a known peptide to vary only a portion of the peptide sequence.

The skilled artisan will also realize that conservative amino acid substitutions may be made in the Sm2 peptide to provide functional variants of the foregoing peptides, i.e., the variants that retain the functional capabilities of the Sm2 peptide. As used herein, a “conservative amino acid substitution” refers to an amino acid substitution which does not alter the relative charge or size characteristics of the peptide in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a Sm2 peptide inhibits TGFβ signaling, one can make conservative amino acid substitutions to the amino acid sequence of the peptide. The substituted peptides can then be tested for one or more of the above-noted functions, in vivo or in vitro. These variants also can be tested for improved stability and are useful, inter alia, in pharmaceutical compositions.

Exemplary functional variants of the Sm2 peptide include conservative amino acid substitutions of peptides having the amino acid sequence of SEQ ID NO:1, SEQ ID NO:6 or SEQ ID NO:7. Such substitutions can be made by a variety of methods known to one of ordinary skill in the art as noted herein. The activity of functional variants of the Sm2 peptide can be tested as disclosed herein.

In another aspect of the invention, binding peptides which bind selectively any of the isolated Sm2 peptides comprising SEQ ID NOs: 1, 6, 7 or functional variants thereof are provided. According to this aspect, the binding peptides bind to an isolated peptide of the invention, including binding to variants thereof. Preferably, the binding peptides bind to a Sm2 peptide, or a variant thereof.

In preferred embodiments, the binding peptide is an antibody or antibody fragment, more preferably, an Fab or F(ab)₂ fragment of an antibody. Typically, the fragment includes a CDR3 region that is selective for the Sm2 peptide. Any of the various types of antibodies can be used for this purpose, including monoclonal antibodies, humanized antibodies and chimeric antibodies.

Antibodies include polyclonal, monoclonal, and chimeric antibodies, prepared, e.g., according to conventional methodology. The antibodies of the present invention are prepared by any of a variety of methods, including administering peptide, variants of peptide, cells expressing the peptide or variants thereof and the like to an animal to induce polyclonal antibodies. The production of monoclonal antibodies is according to techniques well known in the art. Antibodies also may be coupled to specific labeling agents for imaging or to antitumor agents, including, but not limited to, methotrexate, radioiodinated compounds, toxins such as ricin, other cytostatic or cytolytic drugs, and so forth.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. Thus, the invention involves polypeptides of numerous size and type that bind specifically to mutant DOS proteins. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing two or more amino acids to produce peptides of sufficient length. Libraries further can be synthesized of peptides and non-peptide synthetic moieties.

The invention in another aspect also provides pharmaceutical compositions. In one embodiment of this aspect of the invention, the pharmaceutical composition comprises an isolated Sm2 peptide comprising the amino acid sequence of any of SEQ ID NOs:1, 2, 3, 6, 7, 8 or a functional variant thereof, and pharmaceutically acceptable carrier.

In another embodiment, pharmaceutical composition comprises an isolated Sm2 peptide comprising the amino acid sequence of any of SEQ ID NOs:1, 2, 3, 6, 7, 8, or a functional variant thereof, in combination with one or more inhibitors of TβR-I, preferably pyridinylimidazole molecules. Preferably the pyridinylimidazole molecules include those selected from the group consisting of: SB203580, SB202190, SB202474, PD169316, SC68376. These pyridiniylimidazole compounds are low molecular weight compounds that are used as potent inhibitors of tyrosine kinases and serine/threonine kinases and certain ones have been found to inhibit TβR-I kinase at micromolar concentrations [13]. Most of these inhibitors block the ATP-binding sites of the respective enzymes. SB203580, SB202190, SB202474, PD169316, and SC68376 are commercially available from Calbiochem (San Diego, Calif.). The pharmaceutical composition in certain embodiments also includes a pharmaceutically acceptable carrier.

The invention also provides compositions that include two or more pyridinylimidazole molecules selected from the group consisting of: SB203580, SB202190, SB202474, PD169316, and SC68376, and a pharmaceutically acceptable carrier.

Compositions comprising polypeptides that selectively bind a Sm2 peptide, such as monoclonal antibodies described herein, and a pharmaceutically acceptable carrier are also provided in accordance with the invention.

The invention also involves methods for making a medicament that include combining an isolated Sm2 peptide comprising SEQ ID NOs:1, 2, 3, 6, 7 or 8 or a functional variant thereof with a pharmaceutically acceptable carrier to form one or more doses. In some embodiments, the methods include adding one or more small molecule inhibitors of TβR-I, preferably pyridinylimidazole molecules, to the medicament.

The invention also provides methods of inhibiting TGFβ signaling in a cell or a cell extract is provided. The methods generally involve contacting a cell or a cell extract that has TGFβ signaling activity with an isolated Sm2 peptide comprising the amino acid sequence of SEQ ID NOs:1, 2, 3, 6, 7 or 8, or a functional variant thereof, in an effective amount to inhibit TGFβ signaling in the cell or the cell extract. As used herein, a cell can be isolated, part of a tissue (in vivo or in vitro), or part of a culture, such as a cell culture. A “cell extract”, as used herein, includes cell parts, organelles, cell fragments, or protein extracts, such as those prepared by detergent extraction, sonication, homogenization, centrifugation or chromatography.

As used herein, “inhibiting TGFβ signaling” refers to decreasing or slowing the rate of TGFβ signaling including halting or eliminating the TGFβ signaling temporarily or permanently. Inhibiting TGFβ signaling by a Sm2 peptide molecule can be determined, for example, by assaying TGFβ signaling in a sample. For example, such an assay can involve measuring TGFβ signaling in a sample before treatment with a Sm2 peptide, measuring TGFβ signaling in the sample after treatment with a Sm2 peptide, and comparing TGFβ signaling before and after treatment with the Sm2 peptide. A decrease in the TGFβ signaling after treatment with the Sm2 peptide indicates that TGFβ signaling is inhibited by the Sm2 peptide. Observation of no decrease or an increase in the TGFβ signaling after treatment with the Sm2 peptide indicates the TGFβ signaling is not inhibited by the Sm2 peptide.

In other embodiments, the invention provides methods of inhibiting TGFβ signaling in a cell or a cell extract that include contacting a cell or a cell extract having TGFβ signaling activity with an isolated Sm2 peptide comprising the amino acid sequence of SEQ ID NOs:1, 2, 3, 6, 7 or 8, or a functional variant thereof, and one or more small molecule inhibitors of TβR-I (preferably pyridinylimidazole molecules) in an amount effective to inhibit TGFβ signaling in the cell or the cell extract. Preferably the pyridinylimidazole molecule is selected from the group consisting of: SB203580, SB202190, SB202474, PD169316, and SC68376. These methods can be tested in the same manner as the foregoing methods.

In still other embodiments the method of inhibiting TGFβ signaling in a cell or a cell extract involves contacting a cell or a cell extract having TGFβ signaling activity with two or more molecules selected from the group consisting of: SB203580, SB202190, SB202474, PD 169316, and SC68376, in an amount effective to inhibit TGFβ signaling in the cell or the cell extract.

In other embodiments, the methods of inhibiting TGFβ signaling in a cell or a cell extract include contacting a cell having TGFβ signaling activity with a Sm2 binding peptide in an amount effective to inhibit TGFβ signaling in the cell or the cell extract.

The foregoing methods of inhibiting TGFβ signaling in a cell or a cell extract can further include contacting the cell or the cell extract with one or more additional TGFβ signaling inhibitor(s) to inhibit TGFβ signaling in the cell or the cell extract. Examples of TGFβ signaling inhibitor(s) include TGFβ antibodies, ligand-binding proteins (e.g., decorin, biglycan), truncated versions of TGFβ receptor type II and type III, and low molecular weight molecules (e.g., imidazole, oxazole and isoquinoline groups).

The methods of inhibiting TGFβ signaling can be performed in vivo or in vitro. For example, for in vivo treatment, the inhibition can be performed in a tissue in a subject. Alternatively, the inhibition can be performed in vitro or ex vivo (e.g., a cell or a tissue extract, a blood sample, tissue or tumor biopsy). In a particularly preferred embodiment, the treatment can be performed in a biological sample that is a cell-containing sample. Samples of tissue and/or cells for use in the various methods described herein can be obtained through standard methods. Samples can be, for example, surgical samples of any type of tissue or body fluid. Biological samples can be used directly or processed to facilitate analysis. Exemplary biological samples, as used herein, include a cell or population of cells, a cell scraping, a cell extract, a blood sample, a tissue biopsy, including punch biopsy, a tumor biopsy, a bodily fluid, a tissue, or a tissue extract.

The invention also provides methods of inhibiting TβR-I kinase activity -dependent disorders, including Smad2 phosphorylation, in a biological sample. In general, the methods involve contacting a biological sample, such as a cell or a cell extract, having Smad2 phosphorylation activity (i.e., in which Smad2 is phosphorylated) with an isolated Sm2 peptide comprising the amino acid sequence of SEQ ID NOs:1, 2, 3, 6, 7 or 8, or functional variants thereof, in an amount effective to inhibit Smad2 phosphorylation.

As used herein, “inhibiting Smad2 phosphorylation” refers to decreasing or slowing the rate of Smad2 phosphorylation including halting or eliminating the Smad2 phosphorylation temporarily or permanently. Inhibition of TβR-I kinase activity -dependent disorders, including Smad2 phosphorylation, by a Sm2 peptide molecule can be determined, for example, by assaying Smad2 phosphorylation in a sample. Such an assay typically involves measuring the Smad2 phosphorylation in a sample before treatment with a Sm2 peptide, measuring Smad2 phosphorylation in the sample after treatment with a Sm2 peptide, and comparing Smad2 phosphorylation before and after treatment with the Sm2 peptide. A decrease in the Smad2 phosphorylation after treatment with the Sm2 peptide indicates that Smad2 phosphorylation is inhibited by the Sm2 peptide. No decrease or an increase in the Smad2 phosphorylation after treatment with the Sm2 peptide indicates Smad2 phosphorylation is not inhibited by the Sm2 peptide.

As with the methods for inhibiting TGFβ described herein, the method of inhibiting Smad2 phosphorylation may also include contacting a biological sample (in vitro or in vivo) with one or more inhibitors of TβR-I, preferably pyridinylimidazole molecules, more preferably those selected from the group consisting of: SB203580, SB202190, SB202474, PD169316, and SC68376. In some instances, the methods include the use of two or more of the pyridinylimidazole molecules.

In evaluating the success of the foregoing methods of inhibiting Smad2 phosphorylation, the phosphorylation state of Smad2 can be assessed by any method for analyzing phosphoproteins known to one of ordinary skill in the art. In a preferred method, the phosphorylation state of Smad2 is determined by contacting the sample with one or more antibodies that discriminate between phosphorylated Smad2 and non-phosphorylated Smad2.

The invention also provides methods of treating a subject having, or at risk of developing, an TGFβ signaling disorder in which TGFβ signaling is increased relative to normal levels, or in which TGFβ signaling produces physiological effects that are deleterious to the subject. Such methods involve administering to a subject in need of such treatment the isolated Sm2 peptides described herein and/or the pyridinylimidazole molecules described herein, in an amount effective to treat the increased TGFβ signaling disorder. Additional treatments for the specific disorders can be provided to a subject in conjuction with or sequentially with the TGFβ signaling inhibitors described herein.

As used herein, “treating” or “treatment” includes preventing, delaying, abating or arresting the clinical symptoms and/or signs of a disorder or disease, including TGFβ signaling. Treatment also includes reducing or preventing as well as increasing the resistance of a subject to develop a disorder or disease.

As used herein, a subject is a mammal such as a human, non-human primate, cow, horse, pig, sheep, goat, dog, cat, or rodent. In preferred embodiments, the subject is a human.

Exemplary subjects calling for treatment with a Sm2 peptide and/or TβR-I inhibitors (preferably pyridinylimidazole molecules) include subjects having or at risk of having cancer (including solid tumors and non-solid tumors); fibrotic disorders including pulmonary fibrosis and idiopathic myelofibrosis (Le Bousse-Kerdiles et al., Pathol Biol (Paris). 49(2):153-157, 2001); pulmonary edema in acute respiratory distress syndrome (ARDS) (Dhainaut et al., Crit Care Med. 31(4 Suppl):S258-264, 2003) or acute lung injury (Pittet et al., J Clin Invest. 107(12):1537-1544, 2001); Marfan syndrome (Neptune et al., Nat Genet. 33(3):407-411, 2003); Camurati-Engelmann disease (Saito et al., J Biol Chem. 276(15):11469-11472, 2001); hypertrophic obstructive cardiomyopathy (Li et al., J Thorac Cardiovasc Surg. 123(1):89-95, 2002); renal disease including glomerulopathy (Krag et al., Lab Invest. 80(12):1855-1868, 2000) and lupus nephritis (Yamamoto et al., Lab Invest. 80(10):1561-1570, 2000); wound healing, including soft tissue wounds and bone fractures; and immune disorders resulting from the immunosuppressor action of TGFβ. The preferred subjects of the present invention do not have any other indication calling for administration of a Sm2 peptide and/or pyridinylimidazole molecules.

As used herein, a subject having a disorder is a subject with at least one identifiable sign, symptom, or laboratory finding sufficient to make a diagnosis of the disorder in accordance with clinical standards known in the art for identifying such disorders. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a disorder will include identification of a particular cell type present in a sample of a body fluid or tissue obtained from the subject.

As used herein, a subject at risk of having a disorder is a subject with an identifiable risk factor for having the disorder. For example, a subject at risk of having an increased TGFβ signaling disorder can include an individual with a known or suspected exposure to environmental agents associated with an increased risk of having an increased TGFβ signaling disorder. Additionally or alternatively, a subject at risk of having an increased TGFβ signaling disorder can include an individual with a genetic predisposition to developing an increased TGFβ signaling disorder. Yet other examples of a subject at risk of having an increased TGFβ signaling disorder include a subject that previously has been diagnosed with another disorder associated with an increased risk of having an increased TGFβ signaling disorder.

The invention also provides methods of treating a subject having, or at risk of having, an increased TβR-I kinase activity -dependent disorder, including an increased Smad2 phosphorylation disorder. The invention further provides methods of treating a subject having, or at risk of having, a tumor is provided. The methods involve administering to a subject in need of such treatment the isolated Sm2 peptide molecules described herein and/or the pyridinylimidazole molecules described herein, in an amount effective to treat or prevent the increased TβR-I kinase activity -dependent disorder, including an increased Smad2 phosphorylation disorder, or to reduce the size and or growth of the tumor. In some instances, a Sm2 binding peptide also may be administered in an effective amount to treat the disorder or the tumor.

Tumors that can be treated using the methods of the invention include, for example, benign and malignant solid tumors and benign and malignant non-solid tumors. Examples of solid tumors include, but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, and thyroid cancer (including thyroid adenocarcinoma and medullary carcinoma).

Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas). Chronic lymphoid leukemias include, for example, T cell chronic lymphoid leukemias and B cell chronic lymphoid leukemias.

Myeloid disorders include chronic myeloid disorders such as for instance chronic myeloproliferative disorders and myelodysplastic syndrome and acute myeloid leukemia. Chronic myeloproliferative disorders include but are not limited to angiogenic myeloid metaplasia, essential thrombocythemia, chronic myelogenous leukemia, polycythemia vera, and atypical myeloproliferative disorders. Atypical myeloproliferative disorders include atypical chronic myelogenous leukemia, chronic neutrophilic leukemia, mast cell disease, and chronic eosinophilic leukemia.

In some embodiments of the foregoing methods of treating a subject having, or at risk of having, a tumor further comprises administering one or more tumor therapies to treat the tumor. Such therapies include, for example, tumor medicaments, radiation and surgical procedures. As used herein, a “tumor medicament” refers to an agent which is administered to a subject for the purpose of treating a cancer. Various types of medicaments for the treatment of tumors are described herein. For the purpose of this specification, tumor medicaments are classified as chemotherapeutic agents, immunotherapeutic agents, tumor vaccines, hormone therapy, and biological response modifiers.

Tumor medicaments function in a variety of ways. Some cancer medicaments work by targeting physiological mechanisms that are specific to tumor cells. Examples include the targeting of specific genes and their gene products (i.e., proteins primarily) which are mutated in tumors. Such genes include but are not limited to oncogenes (e.g., Ras, Her2, bcl-2), tumor suppressor genes (e.g., EGF, p53, Rb), and cell cycle targets (e.g., CDK4, p21, telomerase). Tumor medicaments can alternately target signal transduction pathways and molecular mechanisms which are altered in tumor cells. Targeting of tumor cells via the epitopes expressed on their cell surface is accomplished through the use of monoclonal antibodies. This latter type of tumor medicament is generally referred to herein as immunotherapy.

Other tumor medicaments target cells other than tumor cells. For example, some medicaments prime the immune system to attack tumor cells (i.e., tumor vaccines). Still other medicaments, called angiogenesis inhibitors, function by attacking the blood supply of solid tumors. Since the most malignant tumors are able to metastasize (i.e., exit the primary tumor site and seed a distal tissue, thereby forming a secondary tumor), medicaments that impede this metastasis are also useful in the treatment of tumor. Angiogenic mediators include basic FGF, VEGF, angiopoietins, angiostatin, endostatin, TNF-α, TNP-470, thrombospondin-1, platelet factor 4, CAI, and certain members of the integrin family of proteins. One category of this type of medicament is a metalloproteinase inhibitor, which inhibits the enzymes used by the tumor cells to exist the primary tumor site and extravasate into another tissue.

Immunotherapeutic agents are medicaments which derive from antibodies or antibody fragments which specifically bind or recognize a tumor antigen. As used herein a tumor antigen is broadly defined as an antigen expressed by a tumor cell. Preferably, the antigen is expressed at the cell surface of the tumor cell. Even more preferably, the antigen is one which is not expressed by normal cells, or at least not expressed to the same level as in tumor cells.

Antibody-based immunotherapies may function by binding to the cell surface of a tumor cell and thereby stimulate the endogenous immune system to attack the tumor cell. Another way in which antibody-based therapy functions is as a delivery system for the specific targeting of toxic substances to tumor cells. Antibodies are usually conjugated to toxins such as ricin (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the tumor and non-specific toxicity to normal cells can be minimized.

In addition to the use of antibodies which are specific for tumor antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many tumor medicaments is to attack the blood vessels feeding a tumor and/or the connective tissues (or stroma) supporting such blood vessels.

The use of immunostimulatory nucleic acids in conjunction with immunotherapeutic agents such as monoclonal antibodies is able to increase long-term survival through a number of mechanisms including significant enhancement of antibody-dependent cellular cytotoxicity (ADCC), activation of NK cells and an increase in IFN-α levels. ADCC can be performed using a immunostimulatory nucleic acid in combination with an antibody specific for a cellular target, such as a tumor cell. When the immunostimulatory nucleic acid is administered to a subject in conjunction with the antibody the subject's immune system is induced to kill the tumor cell. The antibodies useful in the ADCC procedure include antibodies which interact with a cell in the body. Many such antibodies specific for cellular targets have been described in the art and many are commercially available. The nucleic acids when used in combination with monoclonal antibodies serve to reduce the dose of the antibody required to achieve a biological result.

Other types of chemotherapeutic agents which can be used according to the invention include Aminoglutethimide, Asparaginase, Busulfan, Carboplatin, Chlorombucil, Cytarabine HCl, Dactinomycin, Daunorubicin HCl, Estramustine phosphate sodium, Etoposide (VP 16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Tumor vaccines are medicaments which are intended to stimulate an endogenous immune response against tumor cells. Currently produced vaccines predominantly activate the humoral immune system (i.e., the antibody dependent immune response). Other vaccines currently in development are focused on activating the cell-mediated immune system including cytotoxic T lymphocytes which are capable of killing tumor cells. Tumor vaccines generally enhance the presentation of tumor antigens to both antigen presenting cells (e.g., macrophages and dendritic cells) and/or to other immune cells such as T cells, B cells, and NK cells. In some instances, tumor vaccines may be used along with adjuvants, as are well known in the art.

The invention also provides methods of treating a subject having or at risk of having a fibrotic disorder. The methods include administering to a subject in need of such treatment the isolated Sm2 peptide molecules described herein and/or the pyridinylimidazole molecules described herein, in an amount effective to treat or prevent the fibrotic disorder. In some instances, a Sm2 binding peptide also may be administered in an effective amount to treat the fibrotic disorder.

A fibrotic disorder is a disorder which causes the formation of fibrous tissue in the affected organ. Examples of organs that can have a fibrotic disorder include but are not limited to: blood vessels, skin, lung, liver, heart, kidney, pancreas, uterus, gastrointestinal tract, gall bladder, muscle joint, eye, thyroid, and gingiva. Specific fibrotic disorders/diseases include airways fibrosis in asthma, chronic obstructive pulmonary disease (COPD), cystic fibrosis, systemic sclerosis, diabetic nephropathy, and renal tubulointerstitial fibrosis.

In some embodiments of the foregoing methods of treating a subject having or at risk of having a fibrotic disorder further comprises administering one or more fibrotic disorder therapies to treat or prevent the fibrotic disorder. Examples of fibrotic disorder therapies include but are not limited to: penicillamine, azathioprine, methotrexate, cyclophosphamide, recombinant interferon γ, 5-fluorouracil, extracorporeal photochemotherapy, antiplatelet therapy, such as aspirin, dipyridamole, cochicine, chlorambucil, glucocorticoids such as predinipone, reserpine, α-methyldopa, phenoxybenzamine, prazosin, nifedipine, diltiazem, amlodipine, nitroglycerine paste, losartan, ketanserin, fluoxetine, prostacyclin analogue, such as iloprost, epoprostenol (prostacyclin), pentoxifylline, colchicine, p-aminobenzoic acid, vitamin E, relaxin, pilocarpine, cimetidine, ranitidine, H₂ blockers, gastric acid (proton) pump inhibitors, metoclopramide, cisapride, metronidazole, vancomycin, erythromycin, ciprofloxacin, neomycin, tetracycline, propanolol, clonidine, minoxidil, angiotensin-converting enzyme inhibitors, which include captopril, enalapril, and lisinopril.

The invention further includes methods of treating a subject having, or at risk of having, an immunological disorder. The methods include administering to a subject in need of such treatment the isolated Sm2 peptide molecules described herein and/or the pyridinylimidazole molecules described herein, in an amount effective to treat or prevent the immunological disorder. Specific immunological diseases and disorders include autoimmune encephalomyelitis, collagen-induced arthritis, Th1 cell-mediated autoimmunity, and transplantation of organs and tissues. The methods also are useful for treating inflammatory reactions (wound healing, cancer, allergic inflammations).

The foregoing methods of treating a subject having, or at risk of having, an immunological disorder also can include administering one or more immunological disorder therapies to treat or prevent the immunological disorder. Examples of immunological disorder therapies include but are not limited to: immunomodulators such as interferons, interleukins, dimepramol, imiquimod, antibodies against T or B cells (such as anti-CD3 antibody, anti CD-4 antibody, and anti-CD40 antibody), soluble T cell molecules (such as soluble CTLA-4 protein), cyokines, CpG-based and other nucleic acid immunostimulatory molecules and immunoglobulins.

When administered, the therapeutic compositions of the present invention are administered in pharmaceutically acceptable preparations. Such preparations may routinely contain pharmaceutically acceptable concentrations of salt, buffering agents, preservatives, and compatible carriers, and as noted above, optionally other therapeutic agents.

The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The pharmaceutical preparations disclosed herein are prepared in accordance with standard procedures and are administered at dosages that are selected to reduce, prevent or eliminate the condition (See, e.g., Remington's Pharmaceutical Sciences, Mack Publishing Company, Easton, Pa., and Goodman and Gilman's The Pharmaceutical Basis of Therapeutics, Pergamon Press, New York, N.Y., the contents of which are incorporated herein by reference, for a general description of the methods for administering various agents for human therapy).

The pharmaceutically acceptable compositions of the present invention comprise one or more molecules selected from the group consisting of: Sm2 peptides, SB203580, SB202190, SB202474, PD169316, SC68376, and Sm2 binding peptide in association with one or more nontoxic, pharmaceutically acceptable carriers and/or diluents and/or adjuvants and/or excipients, collectively referred to herein as “carrier” materials, and if desired other active ingredients.

The compositions of the present invention may be administered by any route, preferably in the form of a pharmaceutical composition adapted to such a route, and would be dependent on the condition being treated. The compounds and compositions may, for example, be administered orally, intravascularly, intramuscularly, subcutaneously, intraperitoneally, intranasally or topically. Preferred routes of administration include oral and intravenous administration.

For oral administration, the composition may be in the form of, for example, a tablet, capsule, suspension or liquid. The pharmaceutical composition is preferably made in the form of a dosage unit containing a therapeutically effective amount of the active ingredient. Examples of such dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, cellulose, glycine, lactose, maize-starch, mannitol, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The pharmaceutical compositions may also be administered via injection. Formulations for parenteral administration may be in the form of aqueous or non-aqueous isotonic sterile injection solutions or suspensions. These solutions or suspensions may be prepared from sterile powders or granules having one or more of the carriers mentioned for use in the formulations for oral administration. The compounds may be dissolved in polyethylene glycol, propylene glycol, ethanol, corn oil, benzyl alcohol, sodium chloride, sterile water, and/or various buffers.

For topical use the compounds of the present invention may also be prepared in suitable forms to be applied to the skin, or mucus membranes of the nose and throat, and may take the form of creams, ointments, liquid sprays or inhalants, lozenges, or throat paints. Such topical formulations further can include chemical compounds such as dimethylsulfoxide (DMSO) to facilitate surface penetration of the active ingredient. Suitable carriers for topical administration include oil-in-water or water-in-oil emulsions using mineral oils, petrolatum and the like, as well as gels such as hydrogel. Alternative topical formulations include shampoo preparations, oral pastes and mouthwash.

For rectal administration the compounds of the present invention may be administered in the form of suppositories admixed with conventional carriers such as cocoa butter, wax or other glyceride.

Alternatively, the compounds of the present invention may be in powder form for reconstitution at the time of delivery.

Delivery systems for the compositions of the invention can include time-release, delayed release or sustained release delivery systems. Such systems can avoid repeated administrations increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, as used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

The preparations of the invention are administered in effective amounts. An effective amount is that amount of a pharmaceutical preparation or therapeutic agent that alone, or together with further doses, stimulates the desired response, such as preventing the onset of, alleviating the symptoms of, or stopping or slowing the progression of a disorder. In the case of treating a tumor, for example, the desired response is inhibiting the progression of the tumor. This may involve only slowing the progression of the disease temporarily, although more preferably, it involves halting the progression of the disease permanently. These responses can be monitored by routine methods or can be monitored according to diagnostic methods of the invention discussed herein.

The dosage regimen for treating disorder with is selected in accordance with a variety of factors, including the type, age, weight, sex and medical condition of the subject, the severity of the disorder, the route and frequency of administration, the renal and hepatic function of the subject, and the particular compound employed. An ordinarily skilled physician or clinician can readily determine and prescribe the effective amount of the drug required to treat a disorder. In general, dosages are determined in accordance with standard practice for optimizing the correct dosage for treating the disorder.

The dosage regimen can be determined, for example, by following the response to the treatment in terms clinical signs. Examples of such clinical signs are well known in the art, and they include for example the pulse, blood pressure, temperature, and respiratory rate. Harrison's Principles of Internal Medicine, 15th Ed., Fauci A S et al., eds., McGraw-Hill, New York, 2001.

Typically dosages will be dependent upon the condition to be treated. In general, the active agent concentration will range from between 0.01 mg per kg of body weight per day (mg/kg/day) to about 10.0 mg/kg/day. Alternatively, the dosages of the active agent will range from between 0.01 micromole per kg of body weight per day (μmole/kg/day) to about 10 μmole/kg/day. Preferred oral dosages in humans may range from daily total dosages of about 1 -1000 mg/day over the effective treatment period. Preferred intravenous dosages in humans may range from daily total dosages of about 1 - 100 mg/day over the effective treatment period.

In a related aspect, the invention provides a method for forming a medicament that involves placing a therapeutically effective amount of the therapeutic agent in the pharmaceutically acceptable carrier to form one or more doses.

The invention will be more fully understood by reference to the following examples. These examples, however, are merely intended to illustrate the embodiments of the invention and are not to be construed to limit the scope of the invention.

### EXAMPLES

Introduction

Transforming growth factors (TGFβ) is a potent regulator of cell proliferation, differentiation, apoptosis and migration. TGF-β type I receptor (TβR-I), which has intrinsic serine/threonine kinase activity, is a key component in activation of intracellular TGFβ signaling. TβR-I kinase can be regulated by interaction with other proteins or by phosphorylation [12]. Phosphorylation of TβR-I in the juxtamembrane GS-region by TβR-II is crucial for its activation, whereas TβR-I-interacting proteins have a modulatory effect on TGFβ signaling [3-5, 12]. Thus, an efficient way to affect TGFβ signaling, is by affecting the kinase activity of TβR-I.

Low molecular weight compounds have been successfully used as potent inhibitors of tyrosine kinases as well as serine/threonine kinases [13]. Most of these inhibitors block the ATP-binding sites of the respective enzymes. Despite significant similarity of the ATP-binding sites in kinases, it has been possible to develop inhibitors a high degree of selectivity. However, absolute specificities have not been achieved, which complicates their use in treatment of diseases [13, 14]. Another way to achieve specificity is to use inhibitors affecting the substrate-binding site. These inhibitors may have high specificities, e.g. inhibitory peptides for protein kinase C (PKC), p60^{c-src} protein tyrosine kinase, cAMP-dependent protein kinase A (PKA), and myosin light chain kinase [15, 16, 17].

We studied two different classes of TβR-I inhibitors, i.e. compounds interfering with the ATP-binding site of the kinase, and substrate-mimicking peptides. We found that pyridiniylimidazole compounds inhibited TβR-I kinase at micromolar concentrations. A representative compound, SB203580, inhibited in vivo Smad2 phosphorylation by TβR-I, and affected TGFβ-dependent transcriptional activation. Peptides mimicking the TβR-I phosphorylation sites at the C-terminus of Smad2 also inhibited the autophosphorylation of TβR-I and phosphorylation of Smad2 by TβR-I in vitro and in vivo, whereas a similar peptide from Smad5 was without effect. The substrate-mimicking peptide, fused to penetratin (antp), inhibited a TGFβ1-dependent transcriptional response in a luciferase reporter assay, and ligand-dependent growth inhibition of Mv1Lu cells. Thus, substrate-mimetic peptides are a new type of specific inhibitors of the TGFβ signaling in vivo.

Experimental Procedures

Constructs and reagents: To express a GST-TβRI fusion protein, the complete cytoplasmic part (amino acid residues 148-503) of constitutively active TβR-I (T204D) FLAG-tagged at the C-terminus, was inserted into the pGEX4T-1 vector (Pharmacia, Piscataway, N.J.). The protein was produced in E. coli strain β21 and purified using glutathione-Sepharose essentially as described [18]. Purified protein was checked by SDS-PAGE and subsequent Coomassie brilliant blue R-250 staining, and by Western blot using anti-FLAG antibodies (M2; Eastman Kodak, Rochester, N.Y.). GST-Smad3deltaMH1, GT-Smad2 and GST-Smad1 were purified as described elsewhere [18]. SB203580, SB202190, SB202474, SC68375, PD169316, roscovitine, and H7 were obtained from Calbiochem (San Diego, Calif.); PD98059 and staurosporine was obtained from Sigma (St. Louis, Mo.).

Cells: Mv1Lu and COS-7 cells were obtained from ATCC (LGC, Teddington), and cultured in DMEM with 10% FBS.

Peptide synthesis: The antennapedia peptide penetratin (antp) RQIKIWFQNRRMKWKK (SEQ ID NO:5) [19] was used as a control, or was linked with peptides derived from the C-terminus of wild-type Smad2 or from Smad2 and Smad5 in which the phosphorylatable two serine residues were changed to alanine residues; RQIKIWFQNRRMKWKKTQMGSPSVRCSSMS-COOH (antp-Sm2S), (SEQ ID NO:3) RQIKIWFQNRRMKWKKTQMGSPSVRCSAMA-COOH (antp-Sm2A), (SEQ ID NO:2) and RQIKIWFQNRRMKWKKTQMGSPLNPISAVA-COOH (antp-Sm5A) (SEQ ID NO:4). Peptides were synthesized using the Fmoc chemistry as described [20]. Peptides were purified by reverse phase HPLC using a C₁₈ column, and quality and purity of the peptides was confirmed by MALDI-TOF-MS analysis.

In vitro kinase assay: GST-TβRI activity was assayed in vitro using the known TβR-I substrates GST-Smad3deltaMH1, GST-Smad2; GST-Smad1 was used as a control for specificity. The reaction mixture (20 μl) contained 20 mM HEPES (pH 7.4), 10 mM MgCl₂, 2 mM MnCl₂, 1 mM dithiothreitol (DTT), 5 μM ATP plus 0.5 μCi [γ³²P]ATP (Redivue, Amersham Biosciences), and inhibitors at different concentrations. The reaction was initiated by an addition of 0.02 μg purified GST-TβRI, followed by incubation at 22° C. for 20 min. Reaction was terminated by addition of 5 μl of 5× SDS-sample buffer. Samples were subjected to SDS-PAGE followed by analysis in a FujiX2000 phosphorimager. For all in vitro enzyme assays, the percent inhibition was calculated as 100×(1-Aᵢ/A₀), where Aᵢ and A₀ are levels of GST-Smad phosphorylation in the presence and absence of inhibitors, respectively. The IC₅₀ concentration for each compound is defined as a concentration required to inhibit GST-TBRI activity by 50%. Phosphorylated GST-Smad3deltaMH1 and autophosphorylated GST-TβRI were analyzed by tryptic phosphopeptide mapping, as described previously [5].

For in vitro kinase assay of type I and type II TGFβ family receptors, p38α and SAPKα2 kinases, COS-7 cells were transfected with the kinase expression vectors or the control empty pcDNA3 vector using the DEAE-dextran method [25]. Proteins were extracted in a lysis buffer (50 mM Tris-HCl, pH 7.5, 50 mM NaCl, 1% NP-40, 10 μg/mL aprotinin, 0.1 mM PMSF). HA-tagged proteins (ALK1 to ALK7, p38R, SAPKR2) were immunoprecipitated with anti-HA antibody (12CA5; Roche); His₆-tagged proteins (type II receptors) were precipitated with Ni-NTA agarose (Qiagen). The precipitates were washed in extraction buffer, with 20 mM imidazole for Ni-NTA agarose-precipitated proteins, and two times with kinase buffer (20 mM HEPES, pH 7.4, 10 mM MgCl₂, 2 mM MnCl₂, 1 mM DTT). The kinase reaction was initiated by addition of 20 μL of kinase buffer containing 0.5 μCi of [γ-32P]ATP, 5.0 μM ATP, and inhibitory peptides at a final concentration of 10 μM. The reaction was performed at 22° C. for 20 min and terminated by addition of SDS-containing sample buffer and boiling for 5 min. The reaction products were analyzed by SDS-PAGE and exposure in a Fuji X2000 phosphorimager. Equal loading was controlled by immunoblotting aliquots of the samples with a HA probe (Y-11; Santa Cruz) or anti-His antibody (Qiagen).

Growth inhibition assay: Growth inhibition assay with MV1Lu cells, using evaluation of [³H]thymidine incorporation, was performed as described earlier [5, 20]. Briefly, cells were preincubated with peptides for 30 min, and then treated or not with TGFβ1 (0.1 ng/ml) for 24 h. During the last two hours of incubation, [³H]thymidine was added. Radioactivity incorporated into DNA was measured in a scintillation counter. No cytotoxic effects of peptides were observed after 24 h incubation with cells, as evaluated by observation of cells in a microscope.

Luciferase assays: Luciferase assays with CAGA(12)-luc and ARE-luc reporters were performed as described earlier [21, 22].

Activation of Smad2: The effect of inhibitors on the TβR-I mediated phosphorylation of substrates in vivo was determined by immunoblot analysis using specific anti-phosphorylated Smad2 polyclonal antiserum (pS2) [23]. Mv1Lu cells were stimulated with TGFβ 1 in the absence or presence of inhibitors or antennapedia penetratin-fused peptides. Phosphorylation of Smad 2 was visualized by immunoblotting of whole cell extract with pS2 antiserum.

Results

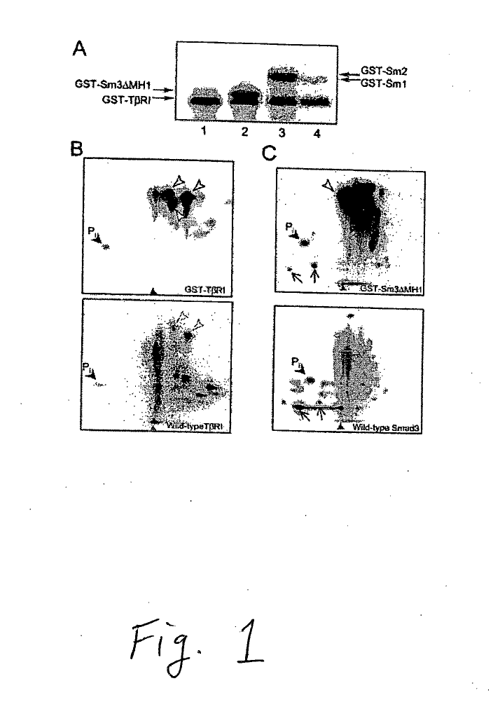

Inhibition of TGF,8 signaling by compounds interfering with the ATP-binding pocket of the TβR-I kinase. To construct an assay for inhibition of the TβR-I kinase, we produced in bacteria the intracellular part of TβR-I receptor (amino acid residues 148-503) fused to GST through its N-terminus (GST-TβR-I). In GST-TβR-I, Thr204 was replaced by an aspartic acid residue, which leads to a constitutive activation of the TβR-I kinase [24]. Purified GST-TβR-I kinase efficiently phosphorylated itself, as well as the C-terminal part of Smad3 and full-length GST-Smad2 as exogenous substrates. Purified GST-TβR-I was subjected to the in vitro kinase assay alone (1) or with purified GST-Smad3deltaMH1 (2), GST-Smad2 (3) or GST-Smad1 (4). Samples were resolved by SDS-PAGE, and labeled proteins were visualized by using a Fuji X2000 phosphorimager. Migration positions of GST-TβR-I, GST-Smad3deltaMH1, GST-Smad2 and GST-Smad1 are shown in FIG. 1A. Consistent with the kinase specificity of TβR-I, no phosphorylation of GST-Smad1 was observed (FIG. 1A).

Autophosphorylated GST-TβR-I, shown in lane 1 of panel A in FIG. 1, was subjected to tryptic digestion and two-dimensional phosphopeptide mapping. Two-dimensional phosphopeptide maps showed similar patterns of autophosphorylation of GST-TβR-I (FIG. 1B) and wild-type full-length TβR-I [5]. Arrowheads indicate position of phosphopeptides, observed both in maps of GST-TβR-I (upper panel) and wild-type TβR-I (lower panel).

GST-Smad3deltaMH1, shown in the lane 2 of panel A, was subjected to two-dimensional phosphopeptide mapping. The phosphopeptide map of GST-Smad3 phosphorylated by GST-TβR-I contained the C-terminal phosphopeptides which are the sites of activating phosphorylation in vivo by TβR-I (FIG. 1 C) [25]. Thus, the produced GST-TβR-I preserved the specificity in phosphorylation of Smad substrates. Arrows show migration positions of the C-terminal peptide with single or double (left spot) phosphorylation. The arrowhead shows migration of the linker-derived peptide with multiple phosphorylations. Sample application points in panels B and C are shown by triangles. Pᵢ indicates migration position of inorganic phosphate.

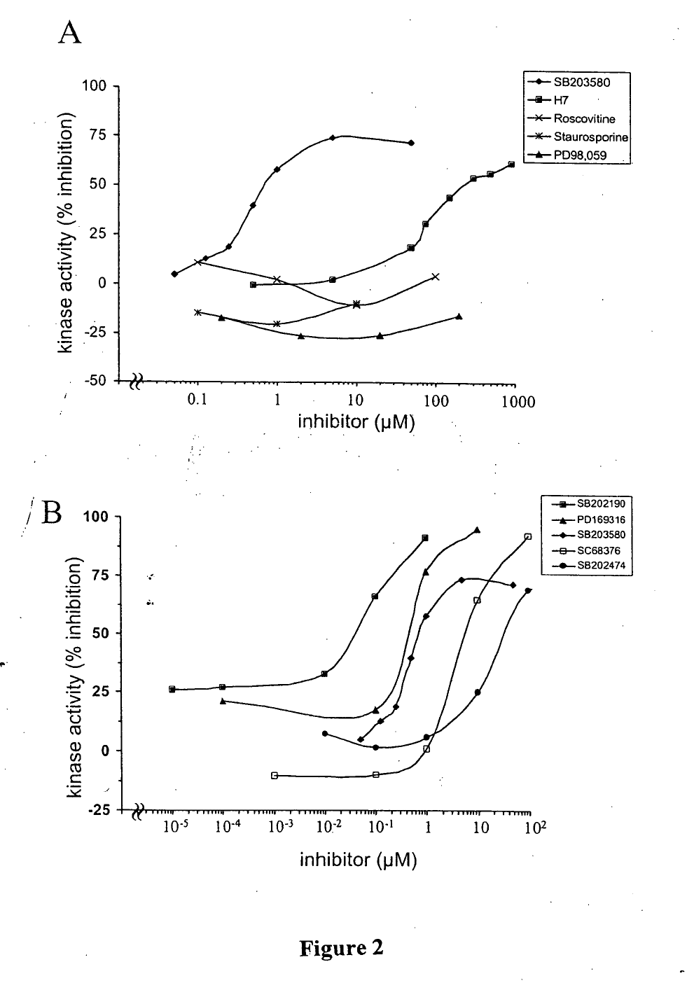

To assess the efficiency of inhibition of TGFβ signaling by compounds interfering with ATP-binding, we studied the effect of known inhibitors of serine/threonine kinases on phosphorylation of substrates by GST-TβR-I. We selected inhibitors which represent five different structural classes, i.e. H7 (isoquinoline group), roscovitine (isopropylpurine group), PD98059 (flavone group), SB203580 (pyridinylimidazole group) and staurosporine. Among them, SB203580 and H7 inhibited TβR-I-dependent phosphorylation of GST-Smad3deltaMH1 in an in vitro kinase assay. The IC₅₀ of SB203580 for inhibition of GST-TβR-I was 700 nM compared to 34 nM for inhibition of the p38 kinase (FIG. 2A). The IC₅₀ value for H7 was 200 μM for inhibition of GST-TβR-I, compared to 97 βM for inhibition of myosin light chain kinase. The other inhibitors, PD98058, roscovitine and staurosporine, did not have an effect on the kinase activity of GST-TβR-I (FIG. 2A). A similarity in the ATP-binding sites of p38 and TβR-I kinases has been reported earlier, and the observed inhibitory effect of SB203580 is in agreement with previous findings [26].

To explore molecular features which can be of importance for the inhibition of TβR-I by pyridinylimidazole compounds, we tested a panel of inhibitors with substitutions in both phenyl rings, as well as in the imidazole ring (FIG. 2B; Table 1). We found that SB202190 has similar IC₅₀ toward GST-TβR-I and p38 kinase, 20 nM compared to 16 nM, respectively. SB202190 has a hydroxyl group at position 4 of the 2-phenyl ring, compared to methylsulfinyl (SB203580) or nitro groups (PD169316). SC68376, which lacks fluor in the 4-fluorophenyl ring, has the imidazole replaced by oxazole and the 2-phenyl ring replaced by a methyl group, compared to SB202190, SB203580 and PD169316, respectively, has increased IC₅₀ for both p38 kinase and GST-TβR-I to 2-5 FM and 5 μM respectively. Interestingly, SB202474 which does not affect p38 kinase, inhibited the kinase activity of GST-TβR-I; however, the IC₅₀ was rather high, 30 μM (Table 1). This suggests that, in contrast to p38 kinase, the 4-fluorophenyl ring is not essential for inhibition of the TβR-I kinase in the in vitro assay and that the size of the 2-imidazole substituent affects the efficiency of the inhibition.

TABLE 1
Inhibition of GST-TβR-I kinase by low molecular weight compounds interfering with ATP-binding site.

| | | IC₅₀ (μM) | | | |
|---|---|---|---|---|---|
| | | GST-TβRI | | p38 MAP | |
| Substance | Structure | in vitro | in vivo | in vitro | in vivo |
| SB203580 | 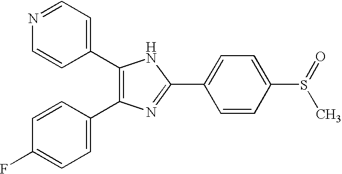 | 0.7 | 10 | 0.034 # | 0.6 # |
| SB202190 | 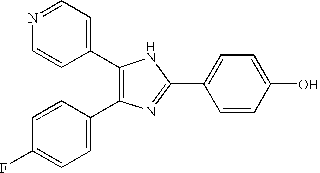 | 0.020 | ND | 0.016 # | 0.35 # |
| PD169316 | 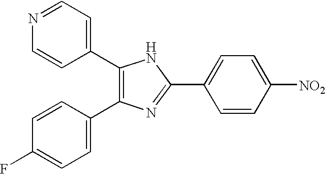 | 0.350 | ND | 0.089 # | ND |
| SC68376 | 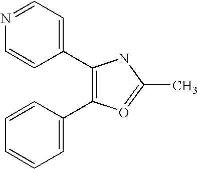 | 5.0 | ND | 2-5 # | ND |
| SB202474 | 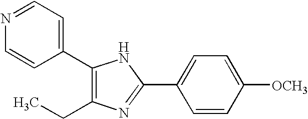 | 30 | ND | ND # | ND |
| H7 | 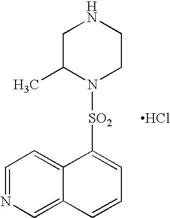 | 200 | ND | ND* | ND |

*Ki for myosin light chain kinase, 97 μM; protein kinase A, 3.0 μM; protein kinase C, 6.0 μM; protein kinase G, 5.8 μM; #
ND - not determined
# - for references see Calbiochem product data sheets

To explore whether SB203580 affects the kinase activity of TβR-I in vivo, we studied phosphorylation of endogenous Smad2 in Mv1Lu cells treated with TGFβI and SB203580. Cells were pre-treated or not with SB203580 before addition of TGFβ1, and the phosphorylation status of Smad2 was evaluated by immunoblotting with antibodies against the two phosphorylated C-terminal serine residues of Smad2 (pS2), which are direct targets of TβR-I kinase [20, 27].

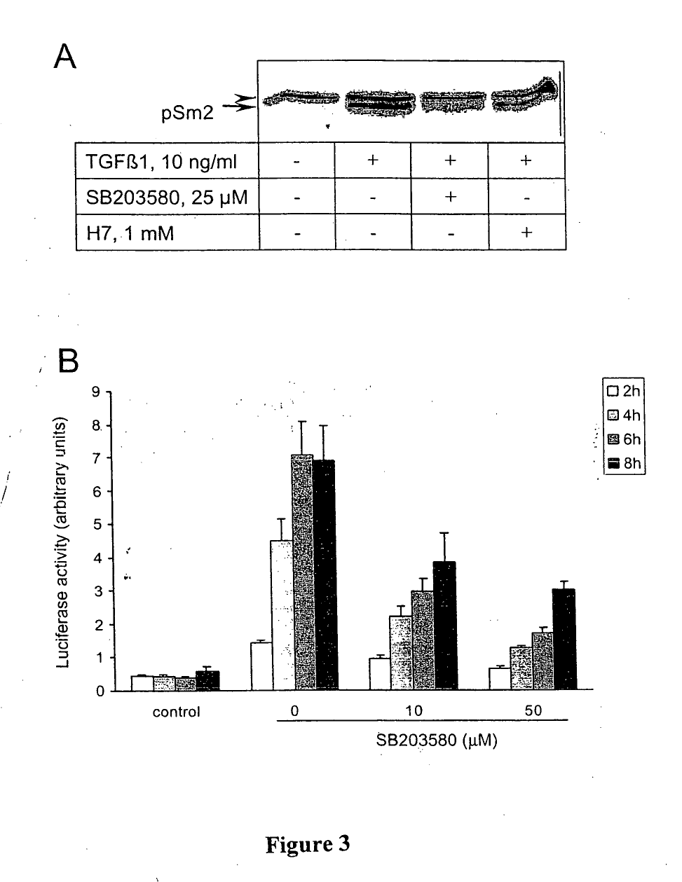

Mv1Lu cells were stimulated with 10 ng/ml of TGFβ 1 for 30 min in the presence or absence of SB203580 and H7, as indicated (FIG. 3A). Smad2 phosphorylated at the C-terminus was detected by immunoblotting of whole cell extract with pS2 antibody. The migration position of phosphorylated Smad2 is shown by arrow, and the arrowhead shows migration of a non-specific band.

We found that pre-treatment of cells with SB203580 at 25 μM significantly reduced TGFβ 1-dependent phosphorylation of Smad2 (FIG. 3A). Quantification of the signals showed that SB203580 inhibited TGFβ 1-induced Smad2 phosphorylation by 57% (data not shown). H7 also inhibited Smad2 phosphorylation, but its effect was weaker than that of SB203580 (FIG. 3A). This is in agreement with the lower efficiency of H7 in inhibition of kinase activity of GST-TβR-I in the in vitro assay (FIG. 2A).

SB203580 at similar concentrations inhibited TGFβ-dependent activation of the luciferase reporter, CAGA(12)-luc, transfected in Mv1Lu cells (FIG. 3B). Mv1Lu cells were transiently transfected with CAGA(12)-luc and Lac-Z (β-gal) reporter plasmids. Cells were stimulated with TGFβ1 (10 ng/ml) for 2, 4, 6 and 8 hours in the presence of SB203580 at indicated concentrations, and luciferase activity was measured. The luciferase activity was normalized to expression of Lac-Z. The inhibitory effect of SB203580 was observed already at a concentration of 10 μM and was most pronounced after 6 hours of TGFβ1 stimulation. Our results show for the first time that in vivo SB203580 inhibits phosphorylation of endogenous Smad2 by TβR-I and affects a TGFβ-specific transcriptional response.

Inhibition of TGFβ signaling by substrate-mimicking peptides. Inhibitors interfering with the ATP-binding site of kinases often have a limited specificity, as ATP-binding pockets of different kinases have significant similarities. In contrast, protein substrate-recognizing surfaces have less similarities, and therefore, can provide specificity for targeting of kinases. To explore the possibility of inhibition of TβR-I kinase by interfering with the binding of substrate to the kinase, we analyzed peptides corresponding to the C-terminus of Smad2 (e.g., antp-Sm2S, SEQ ID NO:3; see Table 2), since the two serine residues at the C-terminus of Smad2 and Smad3 are known as efficient substrates of TβR-I kinase [20, 25, 27]. As phosphorylation of serine residues by the kinase could lead to a quick dissociation of the peptide from the kinase, we also synthesized C-terminal peptides with a substitution of the phosphorylatable serine to alanine residues (antp-Sm2A) (SEQ ID NO:2), as another possible psudosubstrate. A pseudosubstrate occupies the substrate-binding site, but can not be phosphorylated, and therefore does not dissociate from the kinase, and inhibit substrate phosphorylation.

TABLE 2
Peptide Sequences

| | | |
|---|---|---|
| SEQ ID | | |
| NO: | Amino Acid Sequence | Peptide name |

| | | |
|---|---|---|
| 1 | TQMGSPSVRCSAMA | Sm2A |
| 2 | RQIKIWFQNRRMKWKKTQMGSPSVRCSAMA | antp-Sm2A |
| 3 | RQIKIWFQNRRMKWKKTQMGSPSVRCSSMS | antp-Sm2S |
| 4 | RQIKIWFQNRRMKWKKTQMGSPLNPISAVA | antp-Sm5A |
| 5 | RQIKIWFQNRRMKWKK | antp |
| 6 | TQMGSPSVRCSSMS | Sm2S |
| 7 | TQMGSPSVRCSXMX | generic |
| 8 | RQIKIWFQNRRMKWKKTQMGSPSVRCSXMX | generic + |
| | | antp |
| 9 | TQMGSPSIRCSSVS | Smad3 C- |
| | | terminus |

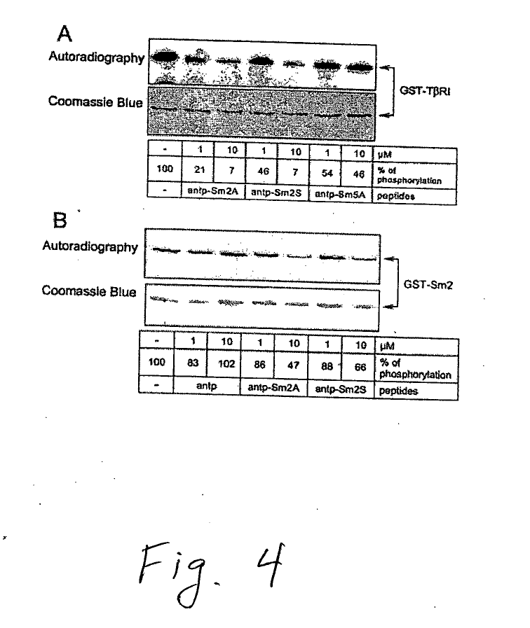

A GST-TβR-I autophosphorylation assay was performed in the absence or presence of antp-Sm2S (SEQ ID NO:3), antp-Sm2A (SEQ ID NO:2) and antp-Sm5A (SEQ ID NO:4) peptides at concentrations of 1 and 10 μM, as indicated (FIG. 4A). Arrow show migration of GST-TβR-I. Smad2 peptides inhibited autophosphorylation of GST-TβR-I in the in vitro kinase assay (FIG. 4A); the antp-Sm2A (SEQ ID NO:2) peptide had a stronger effect than the antp-Sm2S (SEQ ID NO:3) peptide. No or only weak inhibition of autophosphorylation was observed with a peptide mimicking the C-terminus of SmadS, which is not a substrate of TβR-I.

Phosphorylation of GST-Smad2 by GST-TβR-I was performed in an in vitro kinase assay (FIG. 4B). The peptides were added as indicated. Arrow shows migration position of GST-Smad2 (GST-Sm2). Antp-Sm2A (SEQ ID NO:2) peptide also inhibited the phosphorylation at the C-terminus of GST-Smad2, while the antp-Sm2S (SEQ ID NO:3) effect was less pronounced (FIG. 4B). The effect of Sm2 peptides, antp-Sm2A (SEQ ID NO:2) and antp-Sm2S (SEQ ID NO:3) was observed at 10 μM, and maximal inhibition was found at a concentration of 200 μM (data not shown).

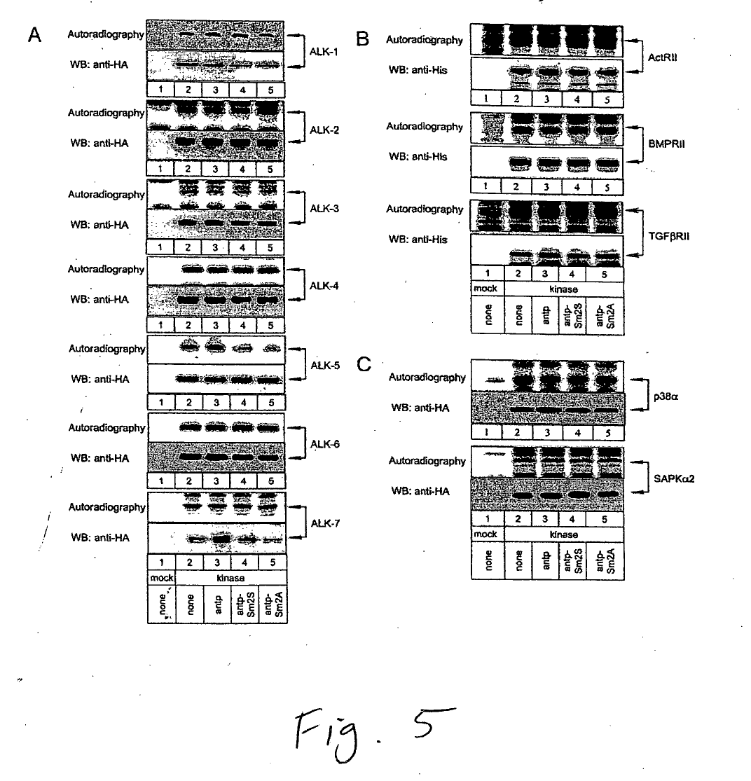

To evaluate whether the substrate-mimicking peptides affect kinases of type I and type II reptors of the TGFβ family, we studied the influence of peptides on authophosphorylation of all known mammalian type I receptors [ALK-1, ActR-I (ALK-2), BMPR-IA (ALK-3), ActR-IB (ALK-4), TβR-I (ALK-5), BMPR-IB (ALK-6), and ALK-7], TβRII, ActR-II, BMPR-II, p38R, and SAPKR2 (FIG. 5). The kinases were expressed in COS-7 cells and purified by using specific anti-tag antibodies or Ni-NTA agarose, and kinase activity was tested in the presence of peptides. We found that antp-Sm2A and antp-Sm2S peptides inhibited the TβR-I kinase, without any effect on the other kinases (FIG. 5), indicating that these peptides are recognized more efficiently by TβR-I than by the other tested kinases.

To evaluate whether the Sm2 peptides also affected TβR-I kinase in vivo, we tested their influence on TGFβ-dependent stimulation of Smad2 phosphorylation. For this assay, we used peptides, which contain in their N-termini a sequence (penetratin, referred to as antp) (SEQ ID NO:5) corresponding to the third helix of the homeodomain of antennapedia, a Drosophila transcription factor [19, 28]; penetratin (antp) (SEQ ID NO:5) or peptides fused to penetratin are efficiently taken up by cells. The molecular mechanism of penetratin-mediated cellular uptake is non-endocytotic and transporter/receptor-independent [19, 28].

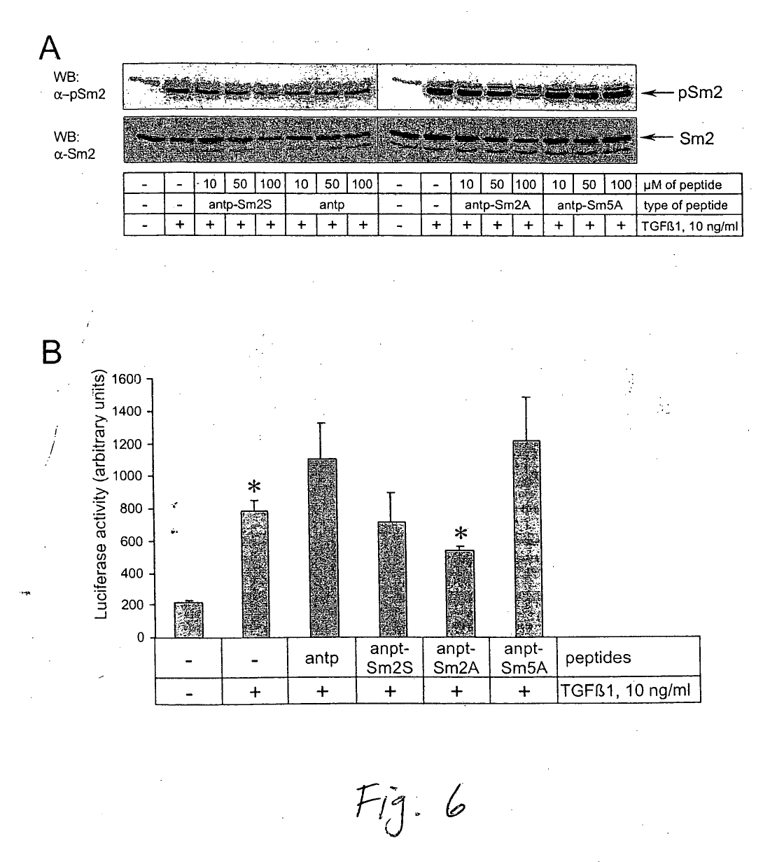

Penetratin (antp)-fused peptides corresponding to the C-terminus of Smad2 with intact C-terminal serine residues (antp-Sm2S) (SEQ ID NO:3) or the same residues replaced by alanine residues (antp-Sm2A) (SEQ ID NO:2), as well as a peptide corresponding to the C-terminus of Smad5 with the serine residues replaced by alanine residues (antp-Sm5A) (SEQ ID NO:4), and a control penetratin peptide (antp) (SEQ ID NO:5), were analyzed. Phosphorylation of endogenous Smad2 was inhibited by pre-treatment of cells with peptides. Mv1Lu cells were incubated with TGFβ1 alone or with antp-Sm2S (SEQ ID NO:3), antp-Sm2A (SEQ ID NO:2), antp-Sm5A (SEQ ID NO:4) and control antp peptides, as indicated. After 15 min of incubation with TGFβ1, phosphorylated Smad2 was detected by immunoblotting of whole cell extract with pSm2 antibody (FIG. 6A, upper panel). To control equal loading, the same membrane was re-probed with anti-Smad2 antibody (FIG. 6A, lower panel). Direct monitoring in a fluorescence microscope of uptake by cells of these peptides conjugated with FITC, confirmed efficient uptake within first 10 minutes of incubation, which was sustained for at least two hours (data not shown). We found that pretreatment of Mv1Lu cells with the antp-Sm2A (SEQ ID NO:2) peptide led to an inhibition of the TGFβ1 -dependent phosphorylation of endogenous Smad2 (FIG. 6A) with 67% of inhibition compared to nontreated cells. Arrows show migration positions of phosphorylated Smad2 (pSm2; upper panel) and Smad2 (Sm2; lower panel).

The antp-Sm2S (SEQ ID NO:3) peptide had weaker affect, and the antp-Sm5A (SEQ ID NO:4) peptide did not have any effect. Effective concentrations of the Smad2 peptides were higher in thess in vivo analyses, compared to the in vitro tests, 50-100 μM compared to 10 μM, respectively (FIG. 4; FIG. 6A); this probably reflects a shorter half-life of the peptides in cells. The effect of antp-Sm2A (SEQ ID NO:2) and antp-Sm2S (SEQ ID NO:3) peptides occurs at the level of receptor kinase activation, since in an in vitro kinase assay with immunoprecipitated constitutively active TβR-I expressed in Mv1Lu cells, antp-Sm2A (SEQ ID NO:2) and antp-Sm2S (SEQ ID NO:3) peptides inhibited autophosphorylation of TβR-I, while antp-Sm5A (SEQ ID NO:4) peptide had only a marginal effect (data not shown). These data were similar to the results of in vitro kinase assay (FIGS. 4 and 5), strongly suggesting that antp-S2A (SEQ ID NO:2) peptide is an inhibitor of TβR-I both in vivo and in vitro.

To explore whether the substrate-mimicking peptides can affect TGFβ-dependent signaling, we performed an assay with a luciferase reporter under control of a Smad2-responsive element. The ARE-luc reporter was transfected in Mv1Lu cells together with xFAST-1, and cells were pre-treated with peptides or not, followed by stimulation with TGFβ1. Mv1Lu cells were transfected with ARE-luc, xFAST-1 and Lac-Z (β-gal) plasmids. Twenty hours after transfection, cells were incubated with TGFβ1 (10 ng/ml) and peptides, as indicated. The final concentration for all peptides was 50 μM. After 18 hours luciferase activity was measured. Pre-treatment of cells with antp-Sm2A (SEQ ID NO:2) inhibited TGFβ1-dependent stimulation up to 31%, while antp-Sm2S (SEQ ID NO:3) had no effect (FIG. 6B). For normalization, Lac-Z activity was measured.

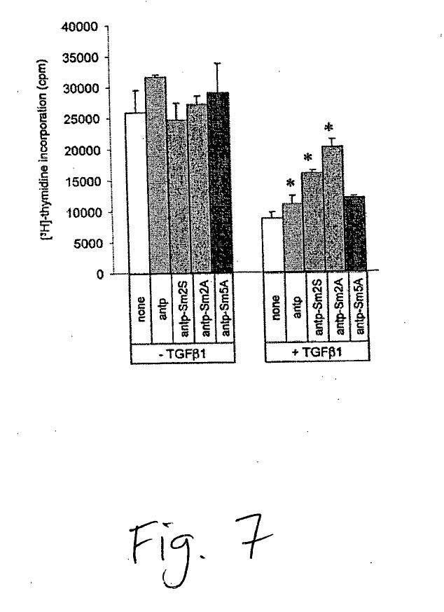

To explore whether the antp-Sm2A (SEQ ID NO:2) peptide can interfere with the TGFβ-dependent effects on cells, we performed an assay with Mv1Lu cells, which are widely used in studies of TGFβ effects and are potently growth-inhibited by TGFβ [29]. Mv1Lu cells were pre-treated with peptides at final concentration 100 μM and incubated with TGFβ1 (0.1 ng/ml) or not for 24 hours, as indicated. [³H]thymidine was added to cells for last two hours of incubation, and radioactivity incorporated into DNA was measured. We found that antp-Sm2S (SEQ ID NO:3) and antp-Sm2A (SEQ ID NO:2) peptides at a concentration of 50 μM reverted to a significant extent the inhibitory action of TGFβ1, while the antp-Sm5A (SEQ ID NO:4) peptide did not have any effect (FIG. 7); for cells treated with antp-Sm2A (SEQ ID NO:2) peptide inhibition in response to TGFβ1 was only 26%, compared to 66% of inhibition for the cells treated with antp peptide only or cells not treated with peptides. In this assay, antp-Sm2A (SEQ ID NO:2) peptide was more efficient than antp-Sm2S (SEQ ID NO:3), suggesting that the peptide has characteristics of a pseudosubstrate. Thus, our data showed that substrate-mimicking peptides specifically inhibit TGFβ-dependent signaling.

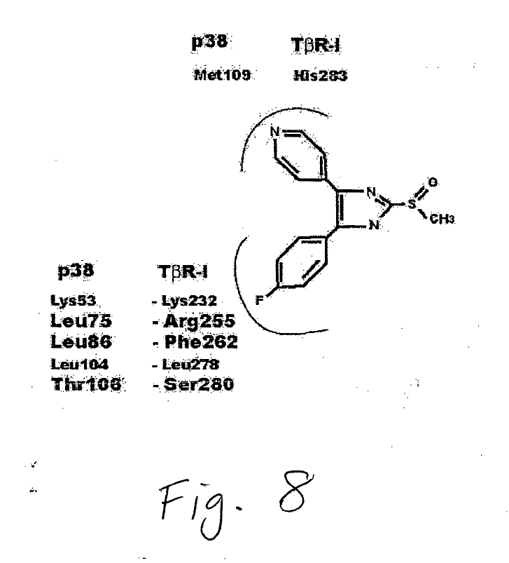

Most of the known kinase inhibitors act through interaction with the ATP-binding site of kinases. Search for specific inhibitors of kinases involved in intracellular signaling, is an important task in the development of drugs, since it may target selected regulatory pathways. In this study, we showed that among the tested serine/threonine kinase inhibitors, compounds of the pyridinylimidazole class inhibited TβR-I kinase in vitro and TGFβ signaling in vivo (FIG. 2; FIG. 3). These compounds are also known as potent inhibitors of the p38 MAP kinase. The similarity between the ATP-binding sites of p38 and TβR-I kinases, has suggested that TβR-I kinase can be sensitive to pyridinylimidazole compounds, and Eyers et al. have shown that SB230580 inhibits autophosphorylation of TβR-I and TBR-II in vitro [26]. Our results provide additional insight into the molecular mechanism whereby TβR-I is inhibited by the pyridinylimidazol compounds. We found that IC₅₀ of different SB203580-related compounds differ in regard to inhibition of TβR-I compared to the p38 kinase (Table 1). The sensitivity of the TβR-I kinase to SB202474, which does not inhibit the p38 kinase, suggests that the 4-fluorophenyl ring is not essential for interaction of the inhibitors with residues in the ATP-binding site of TBR-I kinase. This can be explained by differences in the residues which form bonds with the 4-fluorophenyl ring of inhibitor in p38β vs TβR-I kinases, i.e. Leu75 vs Arg 255; Leu86 vs Phe262, Thr106 vs Ser280, respectively [29, 30] (FIG. 8). A similar observation has been made by Callahan and colleagues [31] . Presence of nitro- or methysulfinyl-groups I on the 2-phenyl ring increased IC₅₀ for TβR-I more than 10 times, compared to the presence of a hydroxyl group, while for p38 kinase the differences were only 2 to 4 times (Table 1). This suggested that the presence of a bulky group in the 2-phenyl ring creates a hindrance, which can lead to a weaker inhibition of the TβR-I kinase. The requirement of a nonbulky group as a 2-imidazole substituent and the dispensability of 4-fluorophenyl suggest that the mechanism of inhibition of TβR-I kinase by SB203580 differs from the mechanisms of p38 inhibition.

The ability of SB203580 to inhibit direct phosphorylation of a substrate, Smad2, by TβR-I in vivo and the potent inhibition of transcriptional activation (FIG. 3) suggest that pyridinylimidazole compounds can provide a basis for development of highly specific TβR-I inhibitors. Recently, Laping et al. [33] and Inman et al. [34] have shown that another pyridinylimidazole analogue, SB431542, can inhibit TGFβ signaling. However, SB431542 lacks absolute specificity as it inhibits also other kinases, i.e., p38R, ALK4, and ALK6. Results by us and others provide information of specificity and potency of various pyridinylimidazole analogues, which will be valuable for further development of highly specific inhibitors.

Inhibitors acting through binding to the ATP-binding site often suffer from low specificity, since ATP-binding sites in all studied kinases share significant similarity. Another potential problem with these inhibitors is that their effects have been determined only for a part of the kinases predicted from the human genome. Therefore, additional kinases can also be affected by these inhibitors. Our data on the inhibition of TβR-I by pyridinylimidazole inhibitors at similar concentration range as for p38 kinases, is an example of this challenge.

To find specific inhibitors, we explored the possibility of affecting the TβR-I kinase by competition with its substrate(s). Sequential phosphorylation of the two C-terminal serine residues in Smad2 and Smad3 by TβR-I is known to trigger intracellular TGFβ signaling [20]. We found that peptides corresponding to the 14 most C-terminal amino acids of Smad2, inhibited efficiently autophosphorylation of TβR-I in vitro, and phosphorylation of Smad2 in vitro and in vivo (FIG. 4; FIG. 6A; data not shown). These peptides did not affect kinase activity of other type I and type II receptors, p38α and SAPKα2 (FIG. 5). The absence of an effect on type II receptors, p38α, SAPKα2, and bone morphogenetic protein signaling-specific type I receptors was expected, since these kinases show a difference in substrate specificity compared to TβR-I. However, the lack of a significant effect on autophosphorylation of ActR-IB and ALK-7, which also phosphorylate Smad2 and Smad3, was unexpected (FIG. 5A). This suggests that type I receptors with similar substrate specificity can have different affinity for the substrate, which may depend on the variability of substrate-kinase interacting surfaces. This issue is currently under investigation.

Similar approaches were used for the generation of specific inhibitors of other kinases, e.g. p60^{c-src}, PKC, Erk, PKA [15-17, 32]. Substrate-mimetic peptides compete at the substrate-binding site, potentially providing higher specificity, compared to competition at the ATP-binding pocket. Moreover, substitution of phosphorylatable residues to non-phosphorylatable, e.g. serine or threonine to alanine residue, significantly increases the affinity of an inhibitory peptide to a kinase. This is due to the inability of the γ-phosphoryl transfer from ATP to an acceptor residue, and therefore, an absence of repulsing force, which is the major contributing factor to the dissociation of phosphorylated substrate from a kinase. Our observation of a higher inhibitory effect of a peptide with phosphorylatable serine residues replaced with alanine residues, compared to the serine-containing peptide, is consistent with the notion that the antp-Sm2A is a pseudosubstrate inhibitor for TβR-I.

Our results show that substrate-mimicking peptides represent a new way of development of specific inhibitors of TGFβ signaling in vivo (FIGS. 6 and 7). Efficient delivery of the peptides into cells in vivo was aided by the use of the antennapedia peptide penetratin. Penetratin provided a basis for new generation of peptide-delivery vectors, and it has allowed specific modulation of signaling processes in different studies [19, 28]. The molecular mechanism of the inhibition of TGFβ signaling comprises blocking of TβR-I kinase activity, as was shown in vitro and in vivo assays (FIGS. 4, 5 and 6A). The inhibitory effect of the peptides was observed both in a short-term (30 min) and in a long-term (18-24 h) assay, i.e. Smad2 phosphorylation and growth inhibition, respectively. This suggests that the peptides were stable in cells and that they did not have a cytotoxic effect during the time of the assays. Thus, the described peptides represent a new class of inhibitors of TGFβ signaling. In combination with the recent developments of methods to introduce peptides into living cells, these peptides will be important tools for regulation of TGFβ signaling.

### References

1. Roberts, A. B., Spom, M. B. Handbook of Experimental Pharmacology. Peptide Growth Factors and their Receptors, 95^{th} Ed. (M. B. Spom and A. B. Roberts, Eds); Springer-Verlag: New York, 419-472 (1990).

2. Massagué, J. Annu. Rev. Biochem,. 67, 753-791 (1998).

3. Miyazono, K., ten Dijke, P., Ichijo, H., and Heldin, C.-H., Adv. Immunol., 55, 181-220 (1994).

4. Wrana, J. L., Attisano, L., Wieser, R., Ventura, F., and Massagué, J., Nature, 370, 341-347, (1994).

5. Souchelnytskyi, S., ten Dijke, P., Miyazono, K., and Heldin, C.-H., EMBO J. 15, 6231-6240, (1996).

6. De Caestecker, M. P., Piek, E., and Roberts, A. B., J. Natl. Cancer Inst., 92, 1388-1402, (2000).

7. Moustakas, A., Souchelnytskyi, S., and Heldin, C.-H., J. Cell Sci., 114, 4359-4369, (2001).

8. Derynk, R., Akhurst, R. J., and Balmain, A., Nat. Genet., 29, 117-129, (2001).

9. Souchelnytskyi, S., Exp. Oncol., 1, 1-6, (2002).

10. Branton, M. H., and Kopp, J. B., Microb. Infect., 1, 1349-1365 (1999).

11. Kulkarni, A. B., Huh, C. G., Becker, D., Geiser, A., Lyght, M., Flanders, K. C., Roberts, A. B., Sporn, M. B., Ward, J. M., and Karlsson, S., Proc. Natl. Acad. Sci., 90, 770-774 (1993) USA.

12. Piek, E., Heldin, C.-H., and ten Dijke, P., FASEB J. 13, 2105-2124 (1999).

13. Lee, J. C., and Adams, J. L., Current Opinion in Biotechnology 6, 657-661.

14. Taylor, S., and Radzio-Andzelm, E., Current Opinion in Chemical Biol. 1, 219-226 (1997).

15. Lou, Q., Leftwich, M. E., McKay, T. R., Salmon, S. E., Rychetsky, L., and Lam, K. S., Canc. Res. 57, 1877-1881.

16. House, C., and Kemp, B. E., Science 238, 1726-1728 (1987).

17. Horiuchi, K. Y., Scherle, P. A., Trzaskos, J. M., and Copeland, R. A. (1998) Biochemistry 37, 8879-8885.

18. Smith, D. B., and Johnson, K. S. (1988) Gen (Amst.) 63, 31-40.

19. Derossi, D., Joliot, A. H. Chassaing, G., and Prochiantz, A. (1994) J. Biol. Chem. 269, 10444-10450.

20. Souchelnytskyi, S., Tamaki, K., Engström, U., Wernstedt, C., ten Dijke, P., and Heldin C.-H. (1997) J. Biol. Chem. 272, 28107-28115.

21. Chen, X., Rubock, M. J., and Witman, M. (1996) Nature 383, 691-696.

22. Denler, S., Itoh, S., Vivien, D., ten Dijke, P., Huet, S., and Gauthier, J.-M. (1998) EMBO J. 17,3091-3100.

23. Piek, E., Westermark, U., Kastemar, M., Heldin, C. H., Vanzoelen, E. J., Nister, M., and ten Dijke, P.(1999) Int. J Cancer 80, 756-763.

24. Weiser, R., Wrana, J., and Massagué, J. (1995) EMBO J. 14, 2199-2208.

25. Yakymovych I., ten Dje, P., Heldin, C.-H., and Souchelnytskyi, S. (2001) FASEB J. 15, 553-555.

26. Eyers, P. A., Craxton, M., Morrice, N., Cohen, P., and Goedert, M. (1998) Cur. Biol. Chemistry and Biology 5, 321-328.

27. Abdollah, S., Macias-Silva, M., Tsukazaki, T., Hayashi, H., Attisano, L., and Wrana, J. L. (1997) J. Biol. Chem. 272, 27678-85.

28. Dunican, D. J., and Doherty, P. (2001) Biopolymers 60, 45-60.

29. Wilson, K. P., McCaffrey, P. G., Hasiao, K., Parzhanisamy, S., Galullo, V., Bemis, G. W., Fitzgibbon, M. J., Caron, P. R., Murcko, M. A., and Su, M. S. S. (1997) Current Biol. Chemistry and Biology 4, 423-431.

30. Huse, M., Chen, Y. G., Massagué, J., and Kuriyan, J. (1999) Cell 96, 425-436.

31. Callahan, J. F., Burgess, J. L., Fornwald, J. A., Gaster, L. M., Harling, J. D., Harrington, F. P., Heer, J., Kwon, C., Lehr, R., Mathur, A., Olson, B. A., Weinstock, J., Laping, N. J. (2002) J Med Chem, 10.1021/jm010493y, January 30.

32. Kemp, B. E., Pearson, R. B., and House, C. M. (1991) Methods Enzymol. 201, 287-304.

33. Laping, N. J., Grygielko, E., Mathur, A., Butter, S., Bomberger, J., Tweed, C., Martin, W., Fornwald, J., Lehr, R., Harling, J., Gaster, L., Callahan, J. F., and Olson, B. A. (2002) Mol. Pharmacol. 62, 58-64.

34. Inman, G. J., Nicolas, F. J., Callahan, J. F., Harling, J. D., Gaster, L. M., Reith, A. D., Laping, N. J., and Hill, C. (2002) Mol. Pharmacol. 62, 65-74.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

1. An isolated peptide that inhibits TGFβ signaling and/or Smad2 phosphorylation, comprising the amino acid sequence of SEQ ID NO:6 or SEQ ID NO:7.

2. The isolated peptide of claim 1, wherein the isolated peptide comprises SEQ ID NO:7:

3. The isolated peptide of claim 2, wherein the isolated peptide consists of SEQ ID NO:7.

4. The isolated peptide of claim 2, wherein the isolated peptide comprises SEQ ID NO:1.

5. The isolated peptide of claim 4, wherein the isolated peptide consists of SEQ ID NO: 1.

6. The isolated peptide of claim 1, wherein the isolated peptide comprises SEQ ID NO:6.

7-17. (canceled)

18. A composition comprising:
the peptide of claim 1 or and
a pharmaceutically acceptable carrier.

19. A composition comprising:
the peptide of claim 1,
one or more molecules selected from the group consisting of: SB203580, SB202190, SB202474, PD169316, and SC68376, and
a pharmaceutically acceptable carrier.

20. A method for making a medicament, comprising:
placing a therapeutically effective amount of the isolated peptide of claim 1 in a pharmaceutically acceptable carrier.

21. A method for making a medicament, comprising:
placing (a) a therapeutically effective amount of the isolated peptide of claim 1 and (b) a therapeutically effective amount of one or more molecules selected from the group consisting of: SB203580, SB202190, SB202474, PD169316, and SC68376, in a pharmaceutically acceptable carrier.

22. A method of inhibiting TGFβ signaling in a cell or cell extract comprising:
contacting a cell or cell extract having TGFβ signaling with an effective amount of a peptide of claim 1 to inhibit TGFβ signaling in the cell or cell extract.

23. A method of inhibiting TGFβ signaling in a cell or cell extract comprising:
contacting a cell or cell extract having TGFβ signaling with an effective amount of (a) a peptide of claim 1 and (b) one or more molecules selected from the group consisting of: SB203580, SB202190, SB202474, PD169316, and SC68376, to inhibit TGFβ signaling in the cell or cell extract.

24-26. (canceled)

27. A method of inhibiting Smad2 phosphorylation in a cell or cell extract comprising:
contacting a cell or cell extract having Smad2 phosphorylation with an effective amount of a peptide of claim 1 to inhibit Smad2 phosphorylation in the cell or cell extract.

28. A method of inhibiting Smad2 phosphorylation in a cell or cell extract comprising:
contacting a cell or cell extract having Smad2 phosphorylation with an effective amount of (a) a peptide of claim 1 and (b) one or more molecules selected from the group consisting of: SB203580, SB202190, SB202474, PD169316, and SC68376, to inhibit Smad2 phosphorylation in the cell or cell extract.

29-31. (canceled)

32. A method of treating a subject having or at risk of having an increased TGFβ signaling disorder comprising:
administering to a subject in need of such treatment an effective amount of a peptide of claim 1 to treat or prevent the increased TGFβ signaling disorder.

33. A method of treating a subject having or at risk of having an increased TGFβ signaling disorder comprising:
administering to a subject in need of such treatment an effective amount of (a) a peptide of claim 1 and (b) one or more molecules selected from the group consisting of: SB203580, SB202190, SB202474, PD169316, and SC68376, to treat the increased TGFβ signaling disorder.

34. (canceled)

35. A method of treating a subject having or at risk of having a disorder that manifests increased TβR-I kinase activity comprising:
administering to a subject in need of such an effective amount of a peptide of claim 1 to treat or prevent the increased TβR-I kinase activity disorder.

36. A method of treating a subject having or at risk of having a disorder that manifests increased TβR-I kinase activity comprising:
administering to a subject in need of such treatment an effective amount of (a) a peptide of claim 1 and (b) one or more molecules selected from the group consisting of: SB203580, SB202190, SB202474, PD169316, and SC68376, in an effective amount to treat the increased TβR-I kinase activity disorder.

37. A method of treating a subject having or at risk of having a disorder that manifests increased Smad2 phosphorylation comprising:
administering to a subject in need of such an effective amount of a peptide of claim 1 to treat or prevent the increased Smad2 phosphorylation disorder.

38. A method of treating a subject having or at risk of having a disorder that manifests increased Smad2 phosphorylation comprising:
administering to a subject in need of such treatment an effective amount of (a) a peptide of claim 1 and (b) one or more molecules selected from the group consisting of: SB203580, SB202190, SB202474, PD169316, and SC68376, in an effective amount to treat the increased Smad2 phosphorylation disorder.
